(19) United States
(12) Patent Application Publication
(10) Pub. No.: US 2019/0262330 A1
(43) Pub. Date: Aug. 29, 2019

(54) Method of Treating Hepatocellular Carcinoma Using N-(4-(6,7-dimethoxyquinolin-4-yloxy)phenyl)-N′-(4-fluorophenyl)cyclopropane-1,1-dicarboxamide, (2S)-hydroxybutanedioate

(71) Applicant: Exelixis, Inc., Alameda, CA (US)

(72) Inventors: Gisela Schwab, Hayward, CA (US); Ann E. Borgman, Menlo Park, CA (US); Colin Hessel, Redwood City, CA (US)

(21) Appl. No.: 16/230,267

(22) Filed: Dec. 21, 2018

Related U.S. Application Data
(60) Provisional application No. 62/609,097, filed on Dec. 21, 2017.
(60) Provisional application No. 62/617,477, filed on Jan. 15, 2018.
(60) Provisional application No. 62/640,166, filed on Mar. 8, 2018.
(60) Provisional application No. 62/640,173, filed on Mar. 8, 2018.

(51) Int. Cl. A61K 31/47; A61P 35/00; A61K 9/20

(52) U.S. Cl. CPC A61K 31/47; A61P 35/00; A61K 9/2009; A61K 9/2018; A61K 9/2054

(57) ABSTRACT

The present disclosure relates to a method of treating liver cancer, particularly hepatocellular carcinoma, using cabozantinib, a kinase inhibitor.

### CROSS-REFERENCE TO RELATED APPLICATIONS

This application claims the benefit of priority to U.S. Provisional Application No. 62/609,097, filed Dec. 21, 2017, and to U.S. Provisional Application No. 62/617,477, filed Jan. 15, 2018, and to U.S. Provisional Application No. 62/640,166, filed Mar. 8, 2018, and to U.S. Provisional Application No. 62/640,173, filed Mar. 8, 2018 the entire contents of which are incorporated herein by reference.

### TECHNICAL FIELD

The present disclosure relates to methods of treating liver cancer, particularly hepatocellular carcinoma, in human patients, particularly to those human patients who have received prior therapy. The method employs cabozantinib, a kinase inhibitor.

### BACKGROUND

Liver cancer is the second leading cause of cancer death worldwide, and mortality from hepatocellular carcinoma (HCC), the most common type of liver cancer, is rising faster than any other cancer in the United States. Ferlay J, Soerjomataram I, Dikshit R, et al. Cancer incidence and mortality worldwide: sources, methods and major patterns in GLOBOCAN 2012. Int J Cancer 2015; 136:E359-86. Ryerson A B, Eheman C R, Altekruse S F, et al. Annual Report to the Nation on the Status of Cancer, 1975-2012, featuring the increasing incidence of liver cancer. Cancer 2016; 122:1312-37. In the majority of cases, diagnosis with or progression to advanced stages of disease requires systemic therapy to delay progression and prolong survival; however, systemic treatment options for advanced disease are limited. Abou-Alfa G K, Jarnagin W, Lowery M, et al. Liver and Bile Duct Cancer. In: Niederhuber J E, Armitage J O, Doroshow J H, Kastan M B, Tepper J E, eds. Abeloff's Clinical Oncology. 5th Ed. Philadelphia, USA: Sanders, 2014: 1373-1396. National Comprehensive Cancer Network. NCCN clinical practice guidelines in oncology. Hepatobiliary Cancers, version 4.2017 (www.nccn.org/professionals/physician_gls/pdf/hepatobiliary.pdf last visited Dec. 13, 2017).

Until recently, the only approved therapy for liver cancer was sorafenib which improves overall survival in patients who have not received prior systemic therapy but results in very few objective responses. Llovet J M, Ricci S, Mazzaferro V, et al. Sorafenib in advanced hepatocellular carcinoma. N Engl J Med 2008; 359:378-90. Cheng A L, Kang Y K, Chen Z, et al. Efficacy and safety of sorafenib in patients in the Asia-Pacific region with advanced hepatocellular carcinoma: a phase III randomised, double-blind, placebo-controlled trial. Lancet Oncol 2009; 10:25-34. For patients previously treated with sorafenib, two therapies have demonstrated clinical benefit. Regorafenib is approved in the United States and Europe based on improved overall survival compared with placebo in sorafenib-tolerant patients who received only sorafenib as prior systemic therapy. Bruix J, Qin S, Merle P, et al. Regorafenib for patients with hepatocellular carcinoma who progressed on sorafenib treatment (RESORCE): a randomised, double-blind, placebo-controlled, phase 3 trial. Lancet 2017; 389:56-66. Nivolumab received accelerated approval in the United States based on objective response rate in a phase 1/2 trial in patients who progressed on or were intolerant to sorafenib. El-Khoueiry A B, Sangro B, Yau T, et al. Nivolumab in patients with advanced hepatocellular carcinoma (CheckMate 040): an open-label, non-comparative, phase 1/2 dose escalation and expansion trial. Lancet 2017; 389:2492-502. Opdivo® [package insert]. Princeton, N.J.: Bristol-Myers Squibb. July 2017.

Despite these advances, treatment outcomes for the majority of patients with advanced hepatocellular carcinoma remain poor, and additional treatment options are urgently needed for patients after progression on front-line therapies.

As a result, a need remains for improved therapies for the treatment of liver cancer, and in particular hepatocellular carcinoma, and using therapies that achieve improvement in the three efficacy endpoints of progression-free survival (PFS), objective response rate, and overall survival in the treatment of hepatocellular carcinoma.

### SUMMARY

These and other needs are met by the present invention, which is directed to a method of treating liver cancer in human patients and, more particularly, to human patients who have received prior therapy. More particularly, the invention is directed to a method of treating heptaocellular carcinoma in human patients, and more particularly to human patients who have received prior therapy. The method employs cabozantinib. The invention is also directed to the use of cabozantinib for treating hepatocellular carcinoma in human patients, and more particularly to human patients who have received prior therapy. The invention is also directed to the use of cabozantinib for treating hepatocellular carcinoma in human patients, and more particularly to human patients who have received prior therapy. The invention is also directed to the use of cabozantinib in the manufacture of a medicament for treating hepatocellular carcinoma in human patients, and more particularly to human patients who have received prior therapy. The invention is also directed to the use of cabozantinib in the manufacture of a medicament for treating hepatocellular carcinoma in human patients.

Another aspect is a method of treating liver cancer in human patients and, more particularly, to human patients who have not received prior therapy, comprising administering to a patient in need of such treatment a therapeutically effective amount of cabozantinib or a pharmaceutically acceptable salt thereof. More particularly, the invention is directed to a method of treating hepatocellular carcinoma in human patients, and more particularly to human patients who have not received prior therapy, with a therapeutically effective amount of cabozantinib or a pharmaceutically acceptable salt thereof.

Cabozantinib is an inhibitor of tyrosine kinases including VEGF receptors 1-3, MET, and AXL, and inhibits tumor growth in murine models of hepatocellular carcinoma. Xiang Q, Chen W, Ren M, et al. Cabozantinib suppresses tumor growth and metastasis in hepatocellular carcinoma by a dual blockade of VEGFR2 and MET. Clin Cancer Res 2014; 20:2959-70. Yakes F M, Chen J, Tan J, et al. Cabozantinib (XL184), a novel MET and VEGFR2 inhibitor, simultaneously suppresses metastasis, angiogenesis, and tumor growth. Mol Cancer Ther 2011; 10:2298-308. In a phase 2 randomized discontinuation trial, cabozantinib showed clinical activity in patients, regardless of whether or not they had been treated with sorafenib for advanced hepatocellular carcinoma; median overall survival was 11.5 months and median progression-free survival was 5.2 months from study entry. Kelley R K, Verslype C, Cohn A L, et al. Cabozantinib in hepatocellular carcinoma: results of a phase 2 placebo-controlled randomized discontinuation study. Ann Oncol 2017; 28:528-34.

A randomized, double-blind, placebo-controlled phase 3 trial (CELESTIAL; NCT01908426) is being conducted to evaluate cabozantinib in previously treated patients with advanced hepatocellular carcinoma. In the study, a total of 707 patients were randomized 2:1 to receive cabozantinib (60 mg once daily) or matching placebo. Eligible patients had received prior sorafenib, progressed following at least one systemic treatment for hepatocellular carcinoma, and may have received up to two prior systemic regimens for advanced hepatocellular carcinoma. The primary endpoint was overall survival. Secondary endpoints were progression-free survival and objective response rate.

The study met the primary endpoint at the second planned interim analysis. Median overall survival was 10.2 months with cabozantinib and 8.0 months with placebo (HR 0.76, 95% CI 0.63-0.92; p=0.0049). Median progression-free survival was 5.2 months with cabozantinib and 1.9 months with placebo (HR 0.44, 95% CI 0.36-0.52; p<0.0001), and the objective response rates were 4% and 0.4%, respectively (p=0.0086). The most common grade 3 or 4 adverse events in the cabozantinib group were palmar-plantar erythrodysesthesia syndrome (17% with cabozantinib vs 0% with placebo), hypertension (16% vs 2%), increased aspartate aminotransferase (12% vs 7%), fatigue (10% vs 4%), and diarrhea (10% vs 2%).

Thus, cabozantinib treatment surprisingly and unexpectedly significantly improves overall survival and progression-free survival compared with placebo and has an acceptable safety profile in patients with previously-treated advanced hepatocellular carcinoma.

Cabozantinib has the structure depicted below.

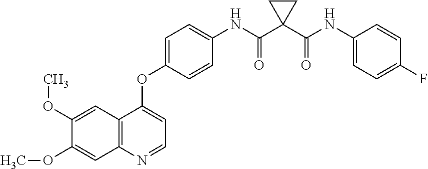 [SMILES: COc1cc2nccc(Oc3ccc(NC(=O)C4(C(=O)Nc5ccc(F)cc5)CC4)cc3)c2cc1OC]

In preferred embodiments, the (S)-malate salt of cabozantinib is administered. Cabozantinib (S)-malate is described chemically as N-(4-(6,7-dimethoxyquinolin-4-yloxy)phenyl)-N′-fluorophenyl)cyclopropane-1,1-dicarboxamide, (2S)-hydroxybutanedioate. The molecular formula is C₂₈H₂₄FN₃O₅.C₄H₆O₅, and the molecular weight is 635.6 Daltons as the malate salt. The chemical structure of cabozantinib (S)-malate salt is depicted below.

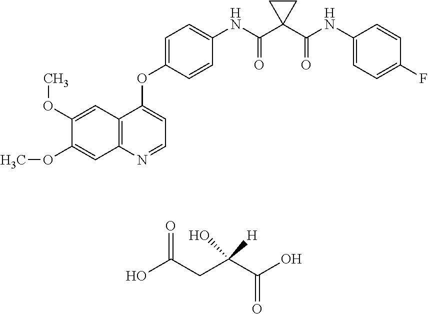 [SMILES: COc1cc2nccc(Oc3ccc(NC(=O)C4(C(=O)Nc5ccc(F)cc5)CC4)cc3)c2cc1OC ; O=C(O)C[C@H](O)C(=O)O]

Cabozantinib as a capsule formulation (COMETRIQ®) has been approved for the treatment of medullary thyroid cancer. Cabozantinib as a tablet formulation (CABOMETYX®) has been approved for the treatment of advanced renal cell carcinoma.

In preferred embodiments, the cabozantinib is administered as CABOMETYX®, which is a tablet formulation of the (S)-malate salt of cabozantinib.

Thus, in one aspect, the invention is directed to a method of treating hepatocellular carcinoma in human patients, comprising administering to the patient an amount of cabozantinib or a pharmaceutically acceptable salt thereof, wherein at least one or more of progression-free survival (PFS), overall survival (OS) and objective response rate (ORR) are extended as compared to patients who have received prior anti-angiogenic therapy. In preferred embodiments of this and other aspects, cabozantinib is administered as cabozantinib (S)-malate.

In another aspect, the invention is directed to a method of treating hepatocellular carcinoma in human patients who have received prior therapy comprising administering to the patient an amount of cabozantinib or a pharmaceutically acceptable salt thereof, wherein at least one or more of progression-free survival (PFS), overall survival (OS) and objective response rate (ORR) are extended as compared to patients who have received prior therapy. In preferred embodiments of this and other aspects, cabozantinib is administered as cabozantinib (S)-malate.

In another aspect the invention is directed to a method of treating hepatocellular carcinoma in human patients who have received prior therapy, comprising administering to the patient an amount of cabozantinib or a pharmaceutically acceptable salt thereof, wherein at least one or more of progression-free survival (PFS), overall survival (OS) and objective response rate (ORR) are extended as compared to patients who have received prior therapy selected from the group consisting of sorafenib, regorafenib, lenvatinib, tivantinib, ramucirumab, Anti-PD-1/PD-L1 therapy, and cytotoxic chemotherapy such as doxorubicin.

A further aspect of the present invention is to provide the use of cabozantinib as the S-malate salt for treating hepatocellular carcinoma in a human patient as described herein. A further aspect of the present invention is to provide the use of cabozantinib as the S-malate salt in the manufacture of a medicament for treating hepatocellular carcinoma in a human patient as described herein.

An additional aspect of the present invention is directed to a method of treating hepatocellular carcinoma, comprising administering to a patient in need of such treatment, 20, 40, or 60 mg of cabozantinib or a pharmaceutically acceptable salt thereof once daily. In one embodiment of this aspect, the patients have received prior therapy, wherein the prior therapy is selected from the group consisting of sorafenib, regorafenib, lenvatinib, tivantinib, ramucirumab, Anti-PD-1/PD-L1 therapy, and doxorubicin. In a further embodiment, the patients have received prior sorafenib therapy. In a further embodiment, the cabozantinib is administered as cabozantinib (S)-malate.

An additional aspect of the present invention is directed to a method of treating hepatocellular carcinoma, comprising administering to a patient in need of such treatment, 20, 40, or 60 mg of cabozantinib or a pharmaceutically acceptable salt thereof once daily, wherein the treatment results in increased overall survival (OS) and one or both of increased progression-free Survival (PFS), and increased overall response rate (ORR). In one embodiment of this aspect, the patients have received prior therapy, wherein the prior therapy is selected from the group consisting of sorafenib, regorafenib, lenvatinib, tivantinib, ramucirumab, Anti-PD-1/PD-L1 therapy, and doxorubicin. In a further embodiment, the patients have received prior sorafenib therapy. In a further embodiment, the cabozantinib is administered as cabozantinib (S)-malate.

An additional aspect of the present invention is directed to a method of treating hepatocellular carcinoma and/or inhibiting proliferation or migration of hepatocellular carcinoma cells, comprising contacting the hepatocellular carcinoma and/or the hepatocellular carcinoma cells with a medicament comprising cabozantinib or a pharmaceutically acceptable salt thereof. In one embodiment of this aspect, the patients have received prior therapy, wherein the prior therapy is selected from the group consisting of sorafenib, regorafenib, lenvatinib, tivantinib, ramucirumab, Anti-PD-1/PD-L1 therapy, and doxorubicin. In a further embodiment, the patients have received prior sorafenib therapy. In a further embodiment, the cabozantinib is administered as cabozantinib (S)-malate.

An additional aspect of the present invention is directed to a method of treating hepatocellular carcinoma in patients who have received prior sorafenib therapy, comprising administering to a patient in need of such treatment, 20, 40, or 60 mg of cabozantinib or a pharmaceutically acceptable salt thereof once daily, wherein the treatment results in increased overall survival (OS) and one or both of increased progression-free survival (PFS), and increased overall response rate (ORR). In one embodiment of this aspect, the cabozantinib is administered as cabozantinib (S)-malate.

In these and other aspects, the hepatocellular carcinoma is metastatic hepatocellular carcinoma, unrescectable hepatocellular carcinoma, refractory hepatocellular carcinoma, or relapsed hepatocellular carcinoma. Furthermore, the hepatocellular carcinoma is resistant to conventional therapy. Examples of such conventional therapies include, but are not limited to, surgery, chemotherapy, radiation therapy, hormonal therapy, biological therapy and immunotherapy.

Additional aspects, embodiments, and advantages of the invention will be set forth in part in the description that follows, and will flow from the description, or can be learned by practice of the invention. The embodiments and advantages of the invention will be realized and attained by means of the elements and combinations particularly pointed out in the appended claims.

It is to be understood that both the foregoing summary and the following detailed description are exemplary and explanatory only and are not restrictive of the invention as claimed.

### BRIEF DESCRIPTION OF THE DRAWINGS

FIG. 1 depicts the framework of the Phase 3 CELESTIAL Study.

FIG. 2 is a summary of patient disposition.

FIGS. 3A and 3B show a comparison of the overall survival and progression-free survival for cabozantinib and a placebo.

FIG. 4 shows the overall survival and progression-free survival in selected subgroups.

FIGS. 5A and 5B show a comparison of the overall survival and progression-free survival for cabozantinib and a placebo for a subgroup of patients whose only prior systemic therapy was sorafenib.

FIG. 6 shows a comparison of the progression-free survival for cabozantinib and everolimus.

FIG. 7 shows a comparison of the overall survival for cabozantinib and everolimus.

FIG. 8 shows a comparison of the progression-free survival for cabozantinib and sunitinib.

FIG. 9 shows a comparison of the overall survival for cabozantinib and sunitinib.

### DETAILED DESCRIPTION

Cabozantinib is administered orally as a pharmaceutical formulation comprising cabozantinib and at least one pharmaceutically acceptable carrier. In one embodiment, cabozantinib is administered as a tablet or capsule formulation. In a further embodiment, cabozantinib is administered as a tablet formulation comprising cabozantinib, particularly cabozantinib as a pharmaceutically acceptable salt, and at least one pharmaceutically acceptable carrier. In a further embodiment, cabozantinib is administered as a tablet formulation comprising cabozantinib (S)-malate, microcrystalline cellulose, anhydrous lactose, hydroxypropyl cellulose, croscarmellose sodium, colloidal silicon dioxide magnesium stearate, and film coating comprising hypromellose, titanium dioxide, triacetin, and iron oxide yellow.

In particular, CABOMETYX® is administered as a tablet comprising cabozantinib (S)-malate, microcrystalline cellulose, anhydrous lactose, hydroxypropyl cellulose, croscarmellose sodium, colloidal silicon dioxide magnesium stearate, and film coating comprising hypromellose, titanium dioxide, triacetin, and iron oxide yellow.

In an embodiment, the cabozantinib (S)-malate is administered as a tablet formulation comprising approximately:
30-32 percent by weight of cabozantinib, (S)-malate salt;
38-40 percent by weight of microcrystalline cellulose;
18-22 percent by weight of lactose;
2-4 percent by weight of hydroxypropyl cellulose;
4-8 percent by weight of croscarmellose sodium;
0.2-0.6 percent by weight of colloidal silicon dioxide;
0.5-1 percent by weight of magnesium stearate; and further comprising:
a film coating material comprising hypromellose, titanium dioxide, triacetin, and iron oxide yellow.

In another embodiment, the cabozantinib (S)-malate is administered as a tablet formulation comprises approximately:
31-32 percent by weight of cabozantinib, (S)-malate salt;
39-40 percent by weight of microcrystalline cellulose;
19-20 percent by weight of lactose;
2.5-3.5 percent by weight of hydroxypropyl cellulose;
5.5-6.5 percent by weight of croscarmellose sodium;
0.25-0.35 percent by weight of colloidal silicon dioxide;
0.7-0.8 percent by weight of magnesium stearate; and further comprising:
3.9-4.1 percent by weight of a film coating material comprising hypromellose, titanium dioxide, triacetin, and iron oxide yellow.

In another embodiment the cabozantinib (S)-malate is administered as a tablet formulation selected from the group consisting of:

| | Theoretical Quantity (mg/unit dose) | | |
|---|---|---|---|
| | 20-mg | 40-mg | 60-mg |
| Ingredient | Tablet* | Tablet* | Tablet* |
| Cabozantinib (S)-malate | 25.34 | 50.69 | 76.03 |
| Microcrystalline Cellulose, PH-102 | 31.08 | 62.16 | 93.24 |
| Lactose Anhydrous, 60M | 15.54 | 31.07 | 46.61 |
| Hydroxypropyl Cellulose, EXF | 2.400 | 4.800 | 7.200 |
| Croscarmellose Sodium | 4.800 | 9.600 | 14.40 |
| Colloidal Silicon Dioxide | 0.2400 | 0.4800 | 0.7200 |
| Magnesium Stearate (Non-Bovine) | 0.6000 | 1.200 | 1.800 |
| Opadry ® Yellow (03K92254) | 3.200 | 6.400 | 9.600 |
| Total Tablet weight | 83.20 | 166.4 | 249.6 |

*Free Base Equivalent

In another embodiment, the cabozantinib (S)-malate is administered as a tablet formulation containing 20, 40, or 60 mg of cabozantinib. 60 mg tablets are yellow film-coated, oval shaped with no score, debossed with “XL” on one side and “60” on the other side of the tablet available in bottles of 30 tablet: NDC 42388-023-26. 40 mg tablets are yellow film-coated, triangle shaped with no score, debossed with “XL” on one side and “40” on the other side of the tablet; available in bottles of 30 tablets: NDC 42388-025-26. 20 mg tablets are yellow film-coated, round shaped with no score, debossed with “XL” on one side and “20” on the other side of the tablet; available in bottles of 30 tablets: NDC 42388-024-26.

CABOMETYX® should be stored at 20° C. to 25° C. (68° F. to 77° F.); excursions are permitted from 15° C. to 30° C. (59° F. to 86° F.) [see USP Controlled Room Temperature].

In a further embodiment, the cabozantinib (S)-malate is administered once daily.

In a further embodiment, the 60 mg of cabozantinib is administered once daily as the CABOMETYX® tablet formulation as described herein.

One aspect is a method of treating hepatocellular carcinoma, comprising administering to a patient in need of such treatment, 20 to 60 mg of cabozantinib or a pharmaceutically acceptable salt thereof once daily.

In one embodiment, 20, 40, or 60 mg of cabozantinib or a pharmaceutically acceptable salt thereof is administered once daily.

In one embodiment, the patient received prior therapy. More particularly, the prior therapy is selected from the group consisting of sorafenib, regorafenib, lenvatinib, tivantinib, ramucirumab, Anti-PD-1/PD-L1 therapy, and doxorubicin.

In one particular embodiment, the patient received prior sorafenib therapy.

In some embodiments, the cabozantinib is administered as cabozantinib (S)-malate.

A further aspect is a method of treating hepatocellular carcinoma, comprising administering to a patient in need of such treatment, 20, 40, or 60 mg of cabozantinib or a pharmaceutically acceptable salt thereof once daily, wherein the treatment results in, as compared to placebo, a median overall survival (OS) of greater than nine months, and one or both of a median progression-free survival (PFS) of greater than 4 months, and a median overall response rate (ORR) of greater than 2 percent.

In one embodiment, the patient received prior therapy. More particularly, the prior therapy is selected from the group consisting of sorafenib, regorafenib, lenvatinib, tivantinib, ramucirumab, Anti-PD-1/PD-L1 therapy, and doxorubicin.

In one particular embodiment, the patient received prior sorafenib therapy.

In one embodiment, the patient has not received prior therapy.

In some embodiments, the cabozantinib is administered as cabozantinib (S)-malate.

Another aspect is a method of treating hepatocellular carcinoma in a patient who received prior sorafenib therapy, comprising administering to a patient in need of such treatment, 20, 40, or 60 mg of cabozantinib or a pharmaceutically acceptable salt thereof once daily, wherein the treatment results in, as compared to placebo, a median overall survival (OS) of greater than nine months, and one or both of a median progression-free survival (PFS) of greater than 4 months, and a median overall response rate (ORR) of greater than 2 months.

An additional aspect of the present invention is directed to a method of treating hepatocellular carcinoma, comprising administering to a patient in need of such treatment, 20, 40, or 60 mg of cabozantinib or a pharmaceutically acceptable salt thereof once daily, wherein the treatment results in overall survival (OS) and one or both of increased progression-free Survival (PFS), and increased overall response rate (ORR). In one embodiment of this aspect, the patients have not received prior therapy. In a further embodiment, the cabozantinib is administered as cabozantinib (S)-malate.

In some embodiments, the cabozantinib is administered as cabozantinib (S)-malate.

A further aspect is a method of treating hepatocellular carcinoma and/or inhibiting the proliferation or migration of hepatocellular carcinoma cells, comprising contacting the hepatocellular carcinoma and/or the hepatocellular carcinoma cells with a medicament comprising cabozantinib or a pharmaceutically acceptable salt thereof.

In one embodiment, the patient received prior therapy. More particularly, the prior therapy is selected from the group consisting of sorafenib, regorafenib, lenvatinib, tivantinib, ramucirumab, Anti-PD-1/PD-L1 therapy, and doxorubicin.

In one particular embodiment, the patient received prior sorafenib therapy.

In some embodiments, the cabozantinib is administered as cabozantinib (S)-malate.

In some embodiments of all aspects, the cabozantinib treatment results in a clinically relevant improvement in one or more markers of disease status and progression, one of more selected from the group consisting of: (i) overall survival; (ii) progression-free survival; (iii) overall response rate; and (iv) reduction in metastatic disease.

In one embodiment, the patient has an overall survival superior or equal to 10 months.

In one embodiment, the patient has an overall survival superior or equal to 11 months.

In one embodiment, the patient has a progression free survival superior or equal to 5 months.

In one embodiment, the patient has a progression free survival superior or equal to 5.5 months.

In one embodiment, the patient has an overall response rate superior or equal to four percent.

In one embodiment, the patient has an overall response rate superior or equal to six percent.

In one embodiment, the patient has an overall survival (OS) of superior or equal to ten months, a progression-free survival (PFS) superior or equal to 5 month, and an overall response rate (ORR) superior or equal to 4 percent.

### Example 1: Cabozantinib versus Placebo in Previously Treated Patients with Advanced Hepatocellular Carcinoma (HCC)

### Method

### Patients

Eligible patients were 18 years of age or older with a pathologic diagnosis of hepatocellular carcinoma not amenable to curative treatment and with Child-Pugh Class A liver function. Patients must have received prior sorafenib and must have progressed following at least one systemic treatment for hepatocellular carcinoma, but could have received up to two prior systemic regimens for advanced hepatocellular carcinoma. Additional inclusion criteria included Eastern Cooperative Oncology Group (ECOG) performance status of 0 or 1, acceptable hematologic parameters, and adequate renal function. Patients could have been treated with prior liver-directed regional therapies as long as they met other eligibility criteria. Patients could not have had prior treatment with cabozantinib or a history of uncontrolled, clinically significant illness.

### Study Design

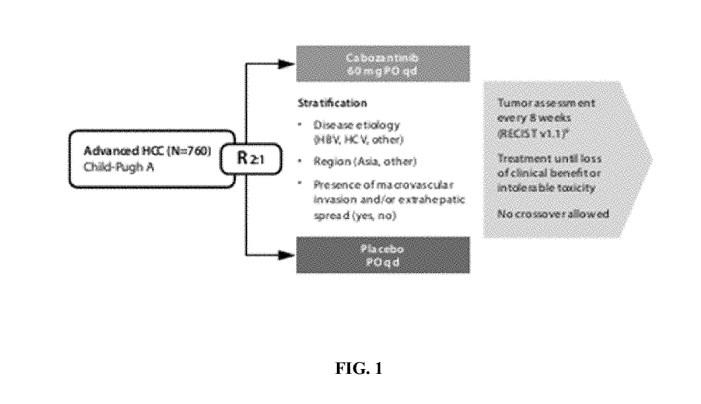

Patients were randomly assigned in a 2:1 ratio to receive either cabozantinib or placebo in this Phase 3 CELESTIAL Study (NCT 01908426), which evaluates cabozantinib compared to placebo in patients with advanced HCC after prior systemic therapy (See FIG. 1). Randomization was stratified by disease etiology (HBV virus [with or without HCV], HCV [without HBV], or other), geographic region (Asia or other), and the presence of extrahepatic spread of disease and/or macrovascular invasion (yes or no).

Patients received either 60 mg cabozantinib orally or matching placebo once per day. Treatment interruptions and dose reductions (to 40 mg and then to 20 mg) were utilized to manage adverse events. Patients received study treatment as long as they experienced clinical benefit as judged by the investigator or until they experienced unacceptable toxicity.

### Endpoints and Assessments

The primary endpoint was overall survival, defined as the time from randomization to death due to any cause, with 90 percent power to detect the hazard ratio of 0.76. 621 events were required among 760 enrolled patients and a two sided log-rank test at 5 percent significance. The inflation of type 1 error was controlled by Lan-DeMets O'Brien-Fleming alpha-spending function.

Secondary efficacy endpoints were progression-free survival, defined as the time from randomization to the earlier of radiographic progression or death due to any cause, and objective response rate. Tumor assessments were performed using computed tomography or magnetic resonance imaging at baseline and every 8 weeks after randomization until the later of 8 weeks after radiographic progression or treatment discontinuation. Tumor response and progression were assessed by the investigator using Response Evaluation Criteria In Solid Tumors (RECIST) version 1.1. Eisenhauer E A, Therasse P, Bogaerts J, et al. New response evaluation criteria in solid tumours: revised RECIST guideline (version 1.1). Eur J Cancer 2009; 45:228-47. Safety was continuously evaluated, and the severity of adverse events was assessed by the investigator using the National Cancer Institute Common Terminology Criteria for Adverse Events, version 4. Progression-free survival was tested by stratified log-rank test; significance level 0.04. Overall response rate was tested by 2-sided Fisher's exact test; significance level Of 0.01.

### Study Oversight

The protocol was approved by the ethics committee or institutional review board at each center, and the study was conducted in accordance with the Good Clinical Practice guidelines and the Declaration of Helsinki. An independent data monitoring committee reviewed safety and efficacy during the study. The study was designed by the first and last authors in collaboration with the sponsor, and the authors and the sponsor were responsible for data collection and analysis. The authors vouch for the fidelity of the study to the protocol and for the accuracy and completeness of the results. Medical writing support was provided by the sponsor. All authors approved submission of the manuscript for publication.

### Statistical Analysis

Up to three event-driven analyses of the primary endpoint of overall survival were planned at approximately 50%, 75%, and 100% information fraction. A sample size of 760 patients with a total of 621 deaths provided the study with 90% power for a two-sided log-rank test at 5% level of significance to detect a hypothesized hazard ratio of 0.76. Assuming a median overall survival of 8.2 months in the placebo group (as observed in the BRISK-PS trial) and exponential distribution, this would correspond to a 32% increase in median overall survival to 10.8 months in the cabozantinib group. Llovet J M, Decaens T, Raoul J L, et al. Brivanib in patients with advanced hepatocellular carcinoma who were intolerant to sorafenib or for whom sorafenib failed: results from the randomized phase III BRISK-PS study. J Clin Oncol 2013; 31:3509-16. DeMets D L, Lan K K. Interim analysis: the alpha spending function approach. Stat Med 1994; 13:1341-52; discussion 53-6.

Inflation of Type 1 error associated with interim analyses was controlled using Lan-DeMets O'Brien-Fleming alpha spending function. If the null hypothesis of no difference in overall survival was rejected at either the first or second interim analysis, testing of secondary endpoints would proceed and subsequent analyses of overall survival would not be performed.

Efficacy was analyzed in all randomized patients by the intent-to-treat principle. Safety was analyzed in all patients who received at least one dose of study treatment. For time-to-event endpoints, hypothesis testing was done using the stratified log-rank test with the stratification factors used for randomization; median durations and associated 95% confidence intervals were estimated using the Kaplan-Meier method. Hazard ratios were estimated using Cox regression models. Hypothesis testing for objective response was done with the Cochran-Mantel-Haenszel method. All subgroup analyses of overall survival and progression-free survival were prespecified except for those based on extrahepatic spread of disease or macrovascular invasion as separate factors or sorafenib as the only prior therapy. For subgroup analyses, no adjustments were made for multiplicity, hazard ratios are unstratified, and confidence intervals are considered descriptive.

### Results

### Patients

From Sep. 15, 2013 through Sep. 18, 2017, 773 patients were randomized at 95 centers in 19 countries. The first interim analysis of overall survival had a data cutoff of Jun. 15, 2016. There were 540 randomized patients representing 52 percent (321 required events). The primary endpoint of overall survival (P=0.0041) did not meet the critical P-value of ≤0.0037. The hazard ratio for overall survival was 0.71. The independent data monitoring committee recommended that the trial continue, and the sponsor remained blinded.

As of the data cutoff date for the second interim analysis of Jun. 1, 2017, 707 patients had been randomized, representing 78 percent (484) of total required events. 470 to receive cabozantinib and 237 to receive placebo, comprising the intent-to-treat population for efficacy analyses. The safety population comprised 704 patients: 467 treated with cabozantinib and 237 treated with placebo. The primary endpoint of overall survival had a P=0.0049 and met the critical P-value of ≤0.021. The hazard ratio for overall survival was 0.76.

As of the data cutoff date, 73 patients (16%) in the cabozantinib group and 26 patients (11%) in the placebo group continued to receive study treatment. The most common reason for study treatment discontinuation was radiographic disease progression. Baseline demographics and clinical characteristics were balanced between the treatment groups (Table 1). All patients had received sorafenib, and 27% had received two prior systemic anticancer regimens for advanced hepatocellular carcinoma.

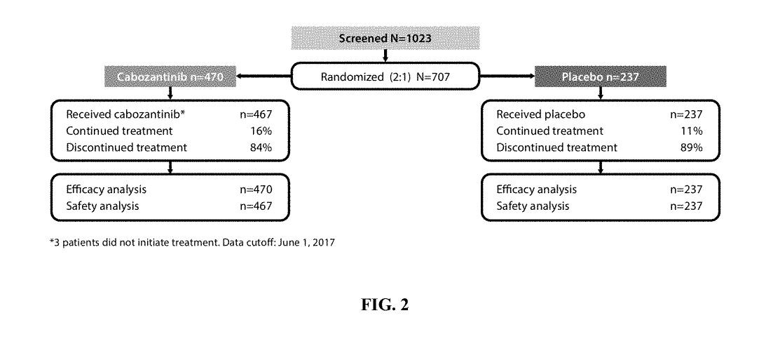

Patient disposition is summarized in FIG. 2. Patient baseline characteristics are summarized in Table 1.

TABLE 1
Baseline Characteristics

| | Cabozantinib | | Placebo | |
|---|---|---|---|---|
| | (N = 470) | | (N = 237) | |
| Age, median (range), years | 64 | (22-86) | 64 | (24-86) |
| Sex, n (%) | | | | |
| Male | 379 | (81) | 202 | (85) |
| Female | 91 | (19) | 35 | (15) |
| Geographic region, n (%) | | | | |
| Asia | 116 | (25) | 59 | (25) |
| Europe | 231 | (49) | 108 | (46) |
| North America (Canada/United States) | 108 | (23) | 59 | (25) |
| Australia/New Zealand | 15 | (3) | 11 | (5) |
| Race, n (%) | | | | |
| White | 264 | (56) | 130 | (55) |
| Asian | 159 | (34) | 82 | (35) |
| Black | 8 | (2) | 11 | (5) |
| Other | 8 | (2) | 2 | (1) |
| Not reported | 31 | (7) | 12 | (5) |
| ECOG performance status, n (%) | | | | |
| 0 | 245 | (52) | 131 | (55) |
| 1 | 224 | (48) | 106 | (45) |
| 2* | 1 | (<1) | 0 | |
| Etiology of disease, n (%)** | | | | |
| HBV (regardless of HCV status) | 178 | (38) | 89 | (38) |
| HCV (regardless of HBV status) | 113 | (24) | 55 | (23) |
| Dual HBV and HCV infection | 8 | (2) | 4 | (2) |
| Alcohol | 112 | (24) | 39 | (16) |
| Nonalcoholic steatohepatitis | 43 | (9) | 23 | (10) |
| Other | 24 | (5) | 16 | (7) |
| Unknown | 75 | (16) | 47 | (20) |
| Child-Pugh class, n (%) | | | | |
| A | 462 | (98) | 235 | (99) |
| B* | 7 | (1) | 2 | (1) |
| Missing | 1 | (<1) | 0 | |
| Extrahepatic spread of disease, n (%) | 369 | (79) | 182 | (77) |
| Macrovascular invasion, n (%) | 129 | (27) | 81 | (34) |
| Extrahepatic spread of disease and/or macrovascular | 398 | (85) | 200 | (84) |
| invasion, n (%) | | | | |
| Sites of disease per investigator at baseline, n (%) | | | | |
| Liver | 395 | (84) | 216 | (91) |
| Bone | 60 | (13) | 34 | (14) |
| Visceral (excluding liver) | 215 | (46) | 105 | (44) |
| Lung | 184 | (39) | 91 | (38) |
| Adrenal gland | 51 | (11) | 24 | (10) |
| Lymph Node | 155 | (33) | 71 | (30) |
| Number of sites (including liver) per investigator | | | | |
| 1 | 144 | (31) | 72 | (30) |
| 2 | 172 | (37) | 91 | (38) |
| ≥3 | 154 | (33) | 74 | (31) |
| Alpha-fetoprotein (ng/mL), n (%) | | | | |
| <400 | 278 | (59) | 136 | (57) |
| ≥400 | 192 | (41) | 101 | (43) |
| Number of prior systemic anticancer regimens for | | | | |
| advanced HCC, n (%) | | | | |
| 0*** | 3 | (1) | 0 | |
| 1 | 335 | (71) | 174 | (73) |
| 2 | 130 | (28) | 62 | (26) |
| ≥3 | 2 | (<1) | 1 | (<1) |
| Prior systemic anticancer therapy, n (%) | | | | |
| Sorafenib | 470 | (100) | 237 | (100) |
| Regorafenib | 6 | (1) | 2 | (1) |
| Lenvatinib | 0 | | 1 | (<1) |
| Tivantinib | 1 | (<1) | 2 | (1) |
| Ramucirumab | 8 | (2) | 1 | (<1) |
| Anti-PD-1/PD-L1 | 14 | (3) | 3 | (1) |
| Cytotoxic chemotherapy | 41 | (9) | 30 | (13) |
| Doxorubicin | 22 | (5) | 10 | (4) |
| Investigational agent | 60 | (13) | 20 | (8) |
| Local liver-directed non-radiation anticancer therapy, | 209 | (44) | 113 | (48) |
| n (%) | | | | |
| Time from initial pathologic diagnosis of HCC to | 1.5 | (0.02-21.9) | 1.3 | (0.03-17.4) |
| randomization, median (range), years**** | | | | |
| Total duration of treatment on prior sorafenib, | 5.3 | (0.3-70.0) | 4.8 | (0.2-76.8) |
| median (range), months | | | | |
| Time from the end of most recent systemic anticancer | 1.4 | (0.0-99.7) | 1.2 | (0.0-69.3) |
| agent for HCC to randomization, median (range), | | | | |
| months | | | | |

HCC, hepatocellular carcinoma
*Although patients were required to have ECOG performance status of 0 or 1 and Child-Pugh class A, a few patients had ECOG performance status of 2 or Child-Pugh class B.
**Etiology per case report form. Patients may have reported more than one disease etiology category.
***Three subjects in the cabozantinib group received prior systemic anticancer therapy that was administered for adjuvant treatment but not for advanced hepatocellular carcinoma treatment.
****Data missing for 1 patient in the cabozantinib group and 2 patients in the placebo group.

Prior therapies of the enrolled patients are further summarized in Table 2.

TABLE 2

| | Cabozantinib | | Placebo | |
|---|---|---|---|---|
| | (N = 470) | | (N = 237) | |
| Number of prior systemic | | | | |
| anticancer regimens for | | | | |
| advanced HCC, n (%) | | | | |
| 0*** | 3 | (1) | 0 | |
| 1 | 335 | (71) | 174 | (73) |
| 2 | 130 | (28) | 62 | (26) |
| ≥3 | 2 | (<1) | 1 | (<1) |
| Prior systemic anticancer | | | | |
| therapy, n (%) | | | | |
| Sorafenib | 470 | (100) | 237 | (100) |
| Regorafenib | 6 | (1) | 2 | (1) |
| Lenvatinib | 0 | | 1 | (<1) |
| Tivantinib | 1 | (<1) | 2 | (1) |
| Ramucirumab | 8 | (2) | 1 | (<1) |
| Anti-PD-1/PD-L1 | 14 | (3) | 3 | (1) |
| Cytotoxic chemotherapy | 41 | (9) | 30 | (13) |
| Doxorubicin | 22 | (5) | 10 | (4) |
| Investigational agent | 60 | (13) | 20 | (8) |
| Local liver-directed non- | 209 | (44) | 113 | (48) |
| radiation anticancer therapy, | | | | |
| n (%) | | | | |
| Time from initial pathologic | 1.5 | (0.02-21.9) | 1.3 | (0.03-17.4) |
| diagnosis of HCC to | | | | |
| randomization, median | | | | |
| (range), years **** | | | | |
| Total duration of treatment on | 5.3 | (0.3-70.0) | 4.8 | (0.2-76.8) |
| prior sorafenib, median | | | | |
| (range), months | | | | |
| Time from the end of most | 1.4 | (0.0-99.7) | 1.2 | (0.0-69.3) |
| recent systemic anticancer | | | | |
| agent for HCC to | | | | |
| randomization, median | | | | |
| (range), months | | | | |

HCC, hepatocellular carcinoma

### Efficacy

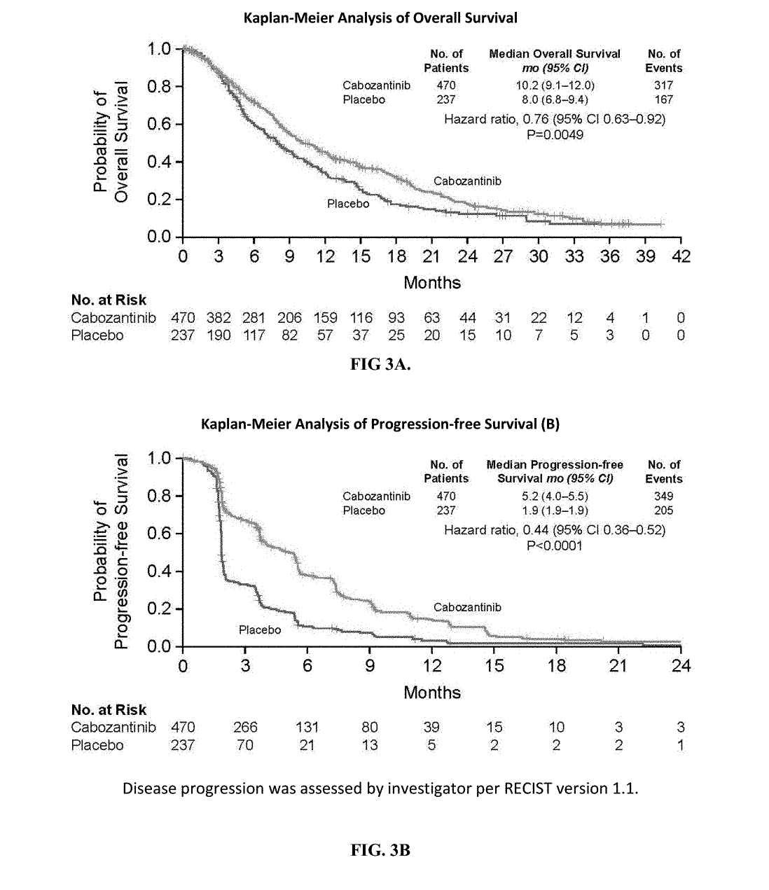

The median overall survival was 10.2 months (95% confidence interval [CI], 9.1 to 12.0) with cabozantinib and 8.0 months (95% CI, 6.8 to 9.4) with placebo (FIG. 3A). The estimated hazard ratio was 0.76 (95% CI, 0.63 to 0.92), and the stratified log-rank p-value was 0.0049, which met the criterion for statistical significance. The primary endpoint was met at the second planned interim analysis (data cutoff date of Jun. 1, 2017) which included 484 deaths, representing 78% of the 621 deaths planned for the prespecified final analysis. The stopping boundary per pre-specified alpha-spending function was p=0.021. Landmark estimates of overall survival by the Kaplan-Meier method at 6, 12, 18, and 24 months showed a higher percentage of patients alive in the cabozantinib group compared with the placebo group at each time point (Table 3).

TABLE 3
Kaplan-Meier Landmark Estimates of Overall Survival

Estimate of % of Patients Alive
(95% CI)

| | Cabozantinib | Placebo |
|---|---|---|
| Landmark | (N = 470) | (N = 237) |
| 6 months | 72 (67-76) | 61 (54-67) |
| 12 months | 46 (41-50) | 34 (28-41) |
| 18 months | 32 (27-37) | 18 (12-24) |
| 24 months | 18 (14-22) | 13 (8-18) |

As of Jun. 1, 2017, 123 patients (26%) in the cabozantinib group and 78 patients (33%) in the placebo group had received subsequent non-radiation systemic or local liver-directed anticancer therapy (Table 4).

TABLE 4
Subsequent Anticancer Therapy

| | Cabozantinib | | Placebo | |
|---|---|---|---|---|
| | (N = 470) | | (N = 237) | |
| Any non-radiation systemic or local liver- | 123 | (26) | 78 | (33) |
| directed anticancer therapy, n (%) | | | | |
| Any systemic anticancer therapy, % | 117 | (25) | 70 | (30) |
| Sorafenib | 19 | (4) | 4 | (2) |
| Regorafenib | 11 | (2) | 3 | (1) |
| Anti-PD-1/PD-L1 | 23 | (5) | 15 | (6) |
| Lenvatinib | 1 | (<1) | 0 | |
| Cytotoxic chemotherapy | 57 | (12) | 40 | (17) |
| Investigational agent | 28 | (6) | 17 | (7) |
| Any non-radiation local liver- | 15 | (3) | 13 | (5) |
| directed anticancer therapy, n (%) | | | | |

These overall survival results are consistent with the findings of the first interim analysis which had a data cutoff date of Jun. 15, 2016 and included 321 patient deaths, representing 52% of the 621 deaths planned for the prespecified final analysis. At that time point, the observed hazard ratio was 0.71 and the p-value was 0.0041, which did not cross the stopping boundary for the first interim analysis (p=0.0037).

The median progression-free survival per RECIST 1.1 per investigator was 5.2 months (95% CI, 4.0 to 5.5) with cabozantinib and 1.9 months (95% CI, 1.9 to 1.9) with placebo. The hazard ratio for progression or death was 0.44 (95% CI, 0.36 to 0.52; stratified logrank p-value<0.0001) (FIG. 3B). The objective response rate per RECIST 1.1 was 4% (18 partial responses out of 470 patients) with cabozantinib and 0.4% (1 partial response out of 237 patients) with placebo (p=0.0086) (Table 5).

TABLE 5
Tumor Response by Investigator

| | Cabozantinib | | Placebo | |
|---|---|---|---|---|
| | (N = 470) | | (N = 237) | |
| Objective response rate (95% | 4 | (2.3-6.0) | 0.4 | (0.0-2.3) |
| CI)^{a, b} | | | | |

Best overall response, n (%)

| Complete response | 0 | | 0 | |
|---|---|---|---|---|
| Partial response | 18 | (4) | 1 | (0.4) |
| Stable disease | 282 | (60) | 78 | (33) |
| Progressive disease | 98 | (21) | 131 | (55) |
| Not evaluable or missing | 72 | (15) | 27 | (11) |

^{a}Confirmed complete and partial responses
^{b}P = 0.0086 (Cochran-Mantel-Haenszel test)

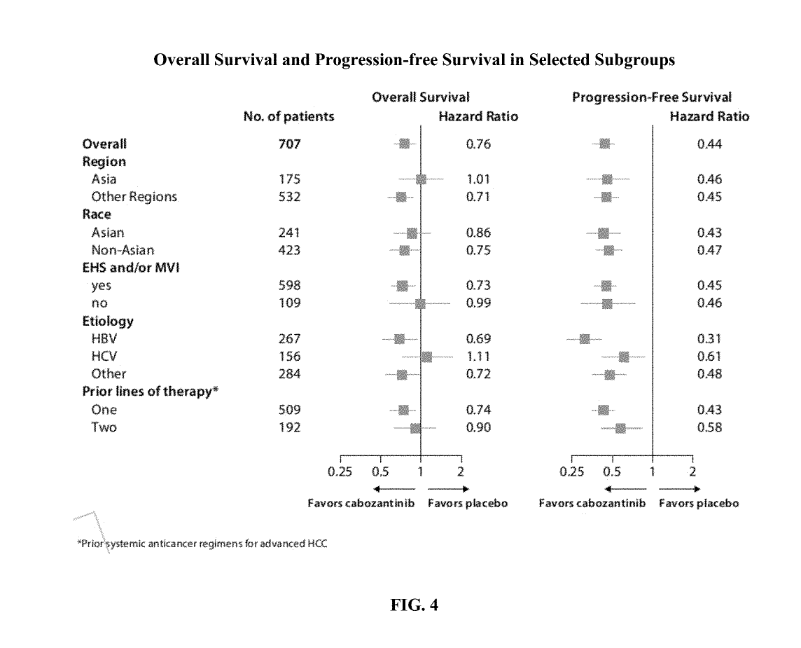

Disease control (partial response or stable disease) was achieved in 64% (300 patients) of the cabozantinib arm compared to 33% (79 patients) of the placebo arm. Subgroup analysis of progression-free survival consistently favored cabozantinib, demonstrating the clinical activity of cabozantinib across various disease etiologies and demographic subgroups (FIG. 4). According to FIG. 4, AFP means alpha-fetoprotein; ECOG PS, means Eastern Cooperative Oncology Group performance status; EHS means extrahepatic spread; MVI means macrovascular invasion. One patient in cabozantinib group had ECOG performance status 2. Thirty-one patients in the cabozantinib group and 12 patients in the placebo group had no race reported. Two patients in the cabozantinib group had unknown MVI status. Three patients in cabozantinib group had no prior regimen for advanced hepatocellular carcinoma, and 2 patients in the cabozantinib group and 1 patient in the placebo group had ≥3 prior regimens. Thus, overall survival in these subgroups was more variable.

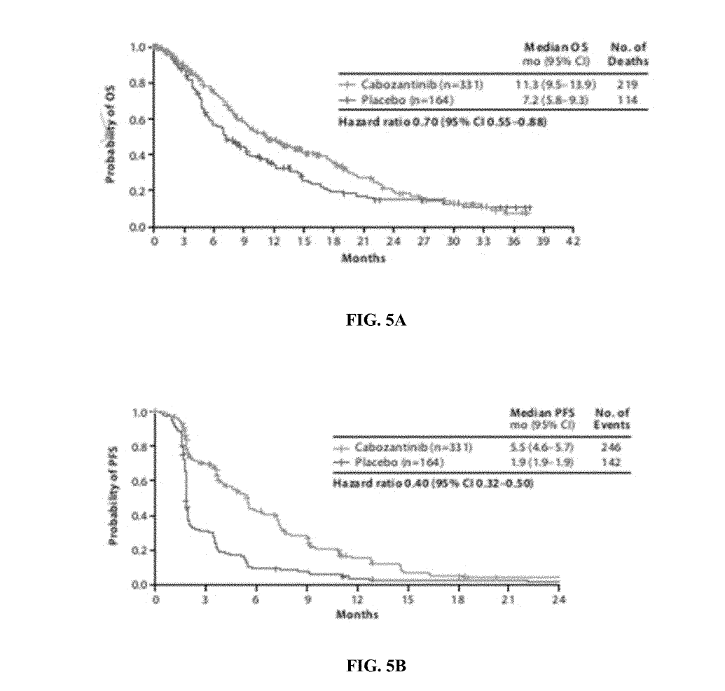

For the subgroup of patients whose only prior systemic therapy was sorafenib (FIG. 5A-B), median overall survival was 11.3 months with cabozantinib and 7.2 months with placebo (HR 0.70; 95% CI, 0.55-0.88), and median progression-free survival was 5.5 months with cabozantinib and 1.9 months with placebo (HR 0.40; 95% CI, 0.32-0.50).

Subsequent anticancer therapy data appears in Table 6.

### Safety

As summarized in Table 7, the median duration of treatment was 3.8 months for cabozantinib-treated patients and 2.0 months for placebo-treated patients.

TABLE 7

| | Cabozantinib | Placebo |
|---|---|---|
| | (N = 470) | (N = 237) |
| Median duration of exposure | 3.8 (0.1 = 37.3) | 2.0 (0.0 = 27.2) |
| (Range), months | | |
| Median average daily dose | 35.8 mg | 58.9 mg |
| Any dose reduction | 62% | 13% |
| Discontinuation due to treatment- | 16% | 3% |
| related adverse event | | |

Dose reductions occurred for 291 patients (62%) treated with cabozantinib and 30 patients (13%) treated with placebo. The median average daily dose was 35.8 mg for cabozantinib and 58.9 mg for placebo, with a median time to first dose reduction of 38 days in the cabozantinib group. The rate of treatment discontinuation due to adverse events considered related to study treatment was 16% (76 patients) in the cabozantinib group and 3% (7 patients) in the placebo group. Adverse events leading to treatment discontinuation in >1.0% of patients in the cabozantinib group were palmar-plantar erythrodysesthesia syndrome, fatigue, decreased appetite, diarrhea, and nausea.

Adverse events of any grade regardless of causality were reported in 99% of cabozantinib-treated patients and 92% of placebo-treated patients, and adverse events of grade 3 or 4 were reported in 68% of cabozantinib-treated patients and 36% of placebo-treated patients (Table 8). The most common grade 3 or 4 adverse events in the cabozantinib group were palmar-plantar erythrodysesthesia syndrome (17% with cabozantinib vs 0% with placebo), hypertension (16% vs 2%), increased aspartate aminotransferase (12% vs 7%), fatigue (10% vs 4%), and diarrhea (10% vs 2%). The most common adverse events of any grade leading to dose reductions with cabozantinib were palmar-plantar erythrodysesthesia syndrome (22%), diarrhea (10%), fatigue (7%), hypertension (7%), and increased aspartate aminotransferase (6%). Serious adverse events were reported for 50% of cabozantinib-treated patients and 37% of placebo-treated patients. Grade 5 adverse events occurring within 30 days of last dose of study treatment were reported for 55 patients (12%) in the cabozantinib group and 28 patients (12%) in the placebo group and were commonly related to disease progression. Grade 5 adverse events considered related to study treatment were reported in 6 patients in the cabozantinib group (one each of hepatic failure, esophagobronchial fistula, portal vein thrombosis, upper gastrointestinal hemorrhage, pulmonary embolism, and hepatorenal syndrome) and one patient in the placebo group (hepatic failure).

### Discussion

This randomized phase 3 trial demonstrates that cabozantinib treatment significantly prolongs survival in patients with previously-treated advanced hepatocellular carcinoma. Median overall survival was 10.2 months with cabozantinib and 8.0 months with placebo, and the hazard ratio was 0.76. Corresponding to this survival benefit, a longer duration of progression-free survival and overall response rate were also improved. Median progression-free survival was 5.2 months with cabozantinib and 1.9 months with placebo with a hazard ratio of 0.44. An improvement in progression-free survival of more than 3 months is consistent with excellent tumor control for patients with progressive hepatocellular carcinoma, and subgroup analyses suggest that cabozantinib has clinical activity across disease etiologies and other baseline characteristics.

Subgroup analyses of overall survival were more variable, with broader confidence intervals. The more consistent improvement in progression-free survival compared with overall survival across subgroups might be due in part to access to subsequent anticancer therapy or prognostic heterogeneity within subgroups. Further analyses will be necessary to help understand these differences.

The safety results for cabozantinib were consistent with results from an earlier phase 2 study in hepatocellular carcinoma and the known safety profile of cabozantinib. Kelley R K, Verslype C, Cohn A L, et al. Cabozantinib in hepatocellular carcinoma: results of a phase 2 placebo-controlled randomized discontinuation study. Ann Oncol 2017; 28:528-34. The most common adverse events were similar to those observed with other VEGFR tyrosine kinase inhibitors in hepatocellular carcinoma. Adverse events were managed with dose modifications and supportive care. Dose reductions occurred for the majority of patients, and the discontinuation rate due to treatment-related adverse events was 16%. The median average daily dose of cabozantinib was 35.8 mg which was similar the value from a phase 3 in advanced renal cell carcinoma (43 mg). Choueiri T K, Escudier B, Powles T, et al. Cabozantinib versus everolimus in advanced renal cell carcinoma (METEOR): final results from a randomised, open-label, phase 3 trial. Lancet Oncol 2016; 17:917-27.

This study was inclusive of a broader patient population than many other phase 3 trials in advanced hepatocellular carcinoma. For example, in the registration trial of regorafenib, patients must have received prior sorafenib but no other prior systemic therapy for hepatocellular carcinoma and must have tolerated a sorafenib dose of at least 400 mg/day. Given the structural similarity of sorafenib and regorafenib, selection of regorafenib-tolerant patients was likely. In contrast, all patients in the CELESTIAL trial were previously treated with sorafenib, but sorafenib tolerance was not required, and over one-quarter of patients had received two systemic anticancer regimens for advanced hepatocellular carcinoma. For the subgroup of patients whose only prior systemic therapy was sorafenib, overall survival and progression-free survival were longer than for the overall study population: median overall survival with cabozantinib was 11.3 months (HR 0.70) and median progression-free survival was 5.5 months (HR 0.40) for this subgroup.

The clinical activity of cabozantinib in hepatocellular carcinoma may reflect its unique target profile relative to other agents evaluated in this disease. MET expression has been shown to increase in tumors after sorafenib exposure in patients with hepatocellular carcinoma, underscoring a possible role for MET in the development of sorafenib resistance. Rimassa L, Abbadessa G, Personeni N, et al. Tumor and circulating biomarkers in patients with second-line hepatocellular carcinoma from the randomized phase II study with tivantinib. Oncotarget 2016; 7:72622-33. Rimassa L, Assenat E, Peck-Radosavljevic M, et al. Second-line tivantinib (ARQ 197) vs placebo in patients (Pts) with MET-high hepatocellular carcinoma (HCC): Results of the METIV-HCC phase III trial. J Clin Oncol 2017; 35: (suppl 15). Abstract 4000.

Tivantinib, an allosteric inhibitor of MET, was evaluated in sorafenib-pretreated patients with high tumor MET expression but had no impact on overall survival or progression-free survival compared with placebo in a MET-high study population. Rimassa L, Assenat E, Peck-Radosavljevic M, et al. Second-line tivantinib (ARQ 197) vs placebo in patients (Pts) with MET-high hepatocellular carcinoma (HCC): Results of the METIV-HCC phase III trial. J Clin Oncol 2017; 35: (suppl 15). Abstract 4000. By inhibiting MET and AXL in addition to VEGF receptors, cabozantinib targets multiple oncogenic and angiogenic pathways, which may provide additional efficacy and help overcome resistance to VEGFR-targeted agents. Gherardi E, Birchmeier W, Birchmeier C, Vande Woude G. Targeting MET in cancer: rationale and progress. Nat Rev Cancer 2012; 12:89-103. Graham D K, DeRyckere D, Davies K D, Earp H S. The TAM family: phosphatidylserine sensing receptor tyrosine kinases gone awry in cancer. Nat Rev Cancer 2014; 14:769-85. Shojaei F, Lee J H, Simmons B H, et al. HGF/c-Met acts as an alternative angiogenic pathway in sunitinib-resistant tumors. Cancer Res 2010; 70:10090-100. Sennino B, Ishiguro-Oonuma T, Wei Y, et al. Suppression of tumor invasion and metastasis by concurrent inhibition of c-Met and VEGF signaling in pancreatic neuroendocrine tumors. Cancer Discov 2012; 2:270-87. Zhou L, Liu XD, Sun M, et al. Targeting MET and AXL overcomes resistance to sunitinib therapy in renal cell carcinoma. Oncogene 2016; 35:2687-97. Xiang Q, Chen W, Ren M, et al. Cabozantinib suppresses tumor growth and metastasis in hepatocellular carcinoma by a dual blockade of VEGFR2 and MET. Clin Cancer Res 2014; 20:2959-70. Firtina Karagonlar Z, Koc D, Iscan E, Erdal E, Atabey N. Elevated hepatocyte growth factor expression as an autocrine c-Met activation mechanism in acquired resistance to sorafenib in hepatocellular carcinoma cells. Cancer Sci 2016; 107:407-16.

Cabozantinib also improved clinical outcomes in advanced renal cell carcinoma after prior antiangiogenic therapy, further supporting a role for targeting MET and AXL in overcoming resistance to VEGF-pathway inhibition. Choueiri T K, Escudier B, Powles T, et al. Cabozantinib versus everolimus in advanced renal cell carcinoma (METEOR): final results from a randomised, open-label, phase 3 trial. Lancet Oncol 2016; 17:917-27. Choueiri T K, Escudier B, Powles T, et al. Cabozantinib versus Everolimus in Advanced Renal-Cell Carcinoma. N Engl J Med 2015; 373:1814-23. Additional biomarker analyses and clinical studies are needed to better understand the role of these targets in the observed clinical activity of cabozantinib in hepatocellular carcinoma as well as to inform future combination strategies.

In conclusion, treatment with cabozantinib, a tyrosine kinase inhibitor that targets MET, VEGF receptors, and AXL, improves overall survival and progression-free survival compared with placebo in patients with previously-treated advanced hepatocellular carcinoma. Adverse events were manageable and consistent with the known safety profile of cabozantinib. Cabozantinib represents a new treatment option for patients with previously-treated advanced hepatocellular carcinoma.

### Example 2: Assessment of Tumor Response, AFP Response, and Time to Progression in the Phase 3 CELESTIAL Trial of Cabozantinib Versus Placebo in Advanced Hepatocellular Carcinoma (HCC)

Background: Cabozantinib inhibits tyrosine kinases including MET, vascular endothelial growth factor receptors, and AXL. In the CELESTIAL trial (NCT01908426), cabozantinib improved overall survival and progression-free survival compared with placebo in patients with previously treated advanced HCC. The study met the primary endpoint, with a median overall survival of 10.2 months with cabozantinib versus 8.0 months with placebo (hazard ratio [HR], 0.76; 95% confidence interval [CI], 0.63-0.92; P=0.0049). Median progression-free survival was 5.2 months with cabozantinib versus 1.9 months with placebo (HR, 0.44; 95% CI, 0.36-0.52; P<0.0001), and the objective response rate was 4% with cabozantinib versus 0.4% with placebo (P=0.0086) per RECIST 1.1. Here, we report a secondary analysis of tumor response including the best percentage change at any timepoint in tumor target lesion size, the best percentage change at any timepoint in serum alpha-fetoprotein (AFP) levels, and time to progression (TTP).

Methods: A total of 707 patients, stratified by disease etiology, geographic region, and extent of disease, were randomized 2:1 to receive cabozantinib 60 mg once daily (n=470) or placebo (n=237). Eligible patients had a pathologic diagnosis of HCC, Child-Pugh score A, and an Eastern Cooperative Oncology Group performance status≤1. Patients must have received prior sorafenib and were allowed up to 2 lines of prior systemic therapy for HCC. Change in the sum of target lesion diameters (SOD) from baseline was determined every 8 weeks by investigator. Best percentage change in SOD was defined as the maximum reduction in SOD at any postbaseline timepoint. Serum AFP levels were measured centrally at baseline and every 8 weeks on the same schedule as tumor assessments. TTP was defined as the time from randomization to radiological progression or clinical deterioration and was determined retrospectively.

Results: Based on the intention-to-treat population, 222 of 470 patients (47%) in the cabozantinib arm and 27 of 237 patients (11%) in the placebo arm had any postbaseline reduction in SOD as best response. Thirty-nine of 470 patients (8%) in the cabozantinib arm and 3 of 237 patients (1%) in the placebo arm had at least one postbaseline tumor assessment with ≥30% reduction in SOD. The percentages of patients in the cabozantinib arm with a ≥30% reduction in postbaseline SOD were 9% (n=26/278) and 7% (n=13/192) among those with a baseline AFP level<400 ng/mL versus ≥400 ng/mL, respectively. Overall, 109 of 470 patients (23%) in the cabozantinib arm and 13 of 237 (5%) in the placebo arm had a ≥50% postbaseline decrease in serum AFP. Median TTP was 5.4 months with cabozantinib versus 1.9 months with placebo (HR, 0.41; 95% CI, 0.34-0.49).

Conclusion: Cabozantinib is associated with improved TTP, higher rates of target lesion regression, and AFP response compared with placebo in patients with previously treated advanced HCC.

### Example 3: Use of Cabozantinib to Treat Hepatocellular Carcinoma

### 1. Indications and Usage

### 1.1. Renal Cell Carcinoma

CABOMETYX is indicated for the treatment of patients with advanced renal cell carcinoma (RCC).

### 1.2. Hepatocellular Carcinoma

CABOMETYX is indicated for the treatment of patients with previously-treated advanced hepatocellular carcinoma (HCC).

### 2. Dosage and Administration

### 2.1 Recommended Dose

Do not substitute CABOMETYX tablets with cabozantinib capsules.

The recommended oral daily dose of CABOMETYX is 60 mg. Do not administer CABOMETYX with food. Instruct patients not to eat for at least 2 hours before and at least 1 hour after taking CABOMETYX. Continue treatment until patient no longer experiences clinical benefit or experiences unacceptable toxicity.

Swallow CABOMETYX tablets whole. Do not crush CABOMETYX tablets.

Do not take a missed dose within 12 hours of the next dose.

Do not ingest foods (e.g., grapefruit, grapefruit juice) or nutritional supplements that are known to inhibit cytochrome P450 during CABOMETYX treatment [see Drug Interactions (7)].

### 2.2 Dosage Adjustments

### For Patients Undergoing Surgery

Stop treatment with CABOMETYX at least 28 days prior to scheduled surgery, including dental surgery [see Warnings and Precautions (5.1)].

### For Adverse Reactions

Management of Grade≤3 adverse reactions may require dose modifications and/or supportive care. If dose reduction is required, a 20 mg decrease from the previously administered dose is recommended. If Grade≤3 adverse reaction is intolerable, withhold CABOMETYX. Withhold CABOMETYX for NCI CTCAE Grade 4 adverse reactions.

If the dose was withheld, upon resolution/improvement (i.e., return to baseline or resolution to Grade 1) of an adverse reaction, reduce the dose as follows:
If previously receiving 60 mg daily dose, resume treatment at 40 mg daily
If previously receiving 40 mg daily dose, resume treatment at 20 mg daily
If previously receiving 20 mg daily dose, resume at 20 mg if tolerated, otherwise, discontinue CABOMETYX

Permanently discontinue CABOMETYX for any of the following:
development of unmanageable fistula or GI perforation
severe hemorrhage
arterial thromboembolic event (e.g., myocardial infarction, cerebral infarction)
hypertensive crisis or severe hypertension despite optimal medical management
nephrotic syndrome
reversible posterior leukoencephalopathy syndrome

### In Patients Concurrently Taking a Strong CYP3A4 Inhibitor

Reduce the daily CABOMETYX dose by 20 mg (for example, from 60 mg to 40 mg daily or from 40 mg to 20 mg daily). Resume the dose that was used prior to initiating the CYP3A4 inhibitor 2 to 3 days after discontinuation of the strong inhibitor [see Drug Interactions (7), Clinical Pharmacology (12.3)]

### In Patients Concurrently Taking a Strong CYP3A4 Inducer

Increase the daily CABOMETYX dose by 20 mg (for example, from 60 mg to 80 mg daily or from 40 mg to 60 mg daily) as tolerated. Resume the dose that was used prior to initiating the CYP3A4 inducer 2 to 3 days after discontinuation of the strong inducer. The daily dose of CABOMETYX should not exceed 80 mg [see Drug Interactions (7), Clinical Pharmacology (12.3)].

### 3 Dosage Forms and Strengths

60 mg CABOMETYX tablets are yellow film-coated, oval shaped with no score, and debossed with “XL” on one side and “60” on the other side.

40 mg CABOMETYX tablets are yellow film-coated, triangle shaped with no score, and debossed with “XL” on one side and “40” on the other side.

20 mg CABOMETYX tablets are yellow film-coated, round with no score, and debossed with “XL” on one side and “20” on the other side.

### 4 Contraindications

None.

### 5 Warnings and Precautions

### 5.1 Hemorrhage

Severe and fatal hemorrhages have occurred with CABOMETYX. The incidence of Grade≥3 hemorrhagic events was 5% in CABOMETYX-treated patients.

Do not administer CABOMETYX to patients that have or are at risk for severe hemorrhage [see Dosage and Administration (2.2)]

### 5.2 GI Perforations and Fistulas

Fistulas were reported in 1% of CABOMETYX-treated patients. Fatal perforations occurred in patients treated with CABOMETYX. Gastrointestinal (GI) perforations were reported in 1% of CABOMETYX-treated patients.

Monitor patients for symptoms of fistulas and perforations, including abscess and sepsis. Discontinue CABOMETYX in patients who experience a fistula which cannot be appropriately managed or a GI perforation.

### 5.3 Thrombotic Events

CABOMETYX treatment results in an increased incidence of thrombotic events. Venous thromboembolism occurred in 7% (including 4% pulmonary embolism) and arterial thromboembolism occurred in 1% of CABOMETYX-treated patients. Fatal thrombotic events occurred in the cabozantinib clinical program.

Discontinue CABOMETYX in patients who develop an acute myocardial infarction or any other arterial thromboembolic complication.

### 5.4 Hypertension and Hypertensive Crisis

CABOMETYX treatment results in an increased incidence of treatment-emergent hypertension, including hypertensive crisis. Hypertension was reported in 36% (17% Grade≥3) of CABOMETYX-treated patients. Monitor blood pressure prior to initiation and regularly during CABOMETYX treatment. Withhold CABOMETYX for hypertension that is not adequately controlled with medical management; when controlled, resume CABOMETYX at a reduced dose. Discontinue CABOMETYX for severe hypertension that cannot be controlled with anti-hypertensive therapy. Discontinue CABOMETYX if there is evidence of hypertensive crisis or severe hypertension despite optimal medical management.

### 5.5 Diarrhea

In RCC studies, diarrhea occurred in 63% of patients treated with CABOMETYX. Grade 3 diarrhea occurred in 11% of patients treated with CABOMETYX. Withhold CABOMETYX in patients who develop intolerable Grade 2 diarrhea or Grade 3-4 diarrhea that cannot be managed with standard antidiarrheal treatments until improvement to Grade 1; resume CABOMETYX at a reduced dose.

### 5.6 Palmar-Plantar Erythrodysesthesia

In RCC studies, palmar-plantar erythrodysesthesia (PPE) occurred in 44% of patients treated with CABOMETYX. Grade 3 PPE occurred in 13% of patients treated with CABOMETYX. Withhold CABOMETYX in patients who develop intolerable Grade 2 PPE or Grade 3 PPE until improvement to Grade 1; resume CABOMETYX at a reduced dose.

### 5.7 Reversible Posterior Leukoencephalopathy Syndrome

Reversible Posterior Leukoencephalopathy Syndrome (RPLS), a syndrome of subcortical vasogenic edema diagnosed by characteristic finding on MRI, occurred in the cabozantinib clinical program. Perform an evaluation for RPLS in any patient presenting with seizures, headache, visual disturbances, confusion or altered mental function. Discontinue CABOMETYX in patients who develop RPLS.

### 5.8 Embryo-Fetal Toxicity

Based on data from animal studies and its mechanism of action, CABOMETYX can cause fetal harm when administered to a pregnant woman. Cabozantinib administration to pregnant animals during organogenesis resulted in embryolethality at exposures below those occurring clinically at the recommended dose, and in increased incidences of skeletal variations in rats and visceral variations and malformations in rabbits. Advise pregnant women of the potential risk to a fetus. Advise females of reproductive potential to use effective contraception during treatment with CABOMETYX and for 4 months after the last dose [See Use in Specific Populations (8.1), (8.3), and Clinical Pharmacology (12.1)].

### 6 Adverse Reactions

The following serious adverse reactions are discussed elsewhere in the label:
Hemorrhage [see Warnings and Precautions (5.1)]
GI Perforations and Fistulas [see Warnings and Precautions (5.2)]
Thrombotic Events [see Warnings and Precautions (5.3)]
Hypertension and Hypertensive Crisis [see Warnings and Precautions (5.4)]
Diarrhea [see Warnings and Precautions (5.5)]
Palmar-plantar erythrodysesthesia [see Warnings and Precautions (5.6)]
Reversible Posterior Leukoencephalopathy Syndrome [see Warnings and Precautions (5.7)]

### 6.1 Clinical Trial Experience

Because clinical trials are conducted under widely varying conditions, adverse reaction rates observed in the clinical trials of a drug cannot be directly compared to rates in the clinical trials of another drug and may not reflect the rates observed in practice.

### Renal Cell Carcinoma

### METEOR

The safety of CABOMETYX was evaluated in METEOR, a randomized, open-label trial in which 331 patients with advanced renal cell carcinoma received 60 mg CABOMETYX and 322 patients received 10 mg everolimus administered daily until disease progression or unacceptable toxicity. Patients on both arms who had disease progression could continue treatment at the discretion of the investigator [see Clinical Studies (14)]. The median duration of treatment was 7.6 months (range 0.3-20.5) for patients receiving CABOMETYX and 4.4 months (range 0.21-18.9) for patients receiving everolimus.

Adverse reactions which occurred in ≥25% of CABOMETYX-treated patients included, in order of decreasing frequency: diarrhea, fatigue, nausea, decreased appetite, palmar-plantar erythrodysesthesia (PPE), hypertension, vomiting, weight decreased, and constipation. Grade 3-4 adverse reactions and laboratory abnormalities which occurred in ≥5% of patients were hypertension, diarrhea, fatigue, PPE, hyponatremia, hypophosphatemia, hypomagnesemia, lymphocytes decreased, anemia, hypokalemia, and GGT increased.

The dose was reduced in 60% of patients receiving CABOMETYX and in 24% of patients receiving everolimus. Twenty percent (20%) of patients received 20 mg CABOMETYX as their lowest dose. The most frequent adverse reactions leading to dose reduction in patients treated with CABOMETYX were: diarrhea, PPE, fatigue, and hypertension. Adverse reactions led to study treatment being held in 70% patients receiving CABOMETYX and in 59% patients receiving everolimus. Adverse reactions led to study treatment discontinuation in 10% of patients receiving CABOMETYX and in 10% of patients receiving everolimus. The most frequent adverse reactions leading to permanent discontinuation in patients treated with CABOMETYX were decreased appetite (2%) and fatigue (1%).

TABLE 9
Adverse Reactions Occurring in ≥10%
Patients Who Received CABOMETYX in METEOR

| | CABOMETYX | | Everolimus | |
|---|---|---|---|---|
| | (n = 331) ¹ | | (n = 322) | |
| | All | Grade | All | Grade |
| | Grades² | 3-4 | Grades² | 3-4 |
| Adverse Reaction | Percentage (%) of Patients | | | |
| Gastrointestinal Disorders | | | | |
| Diarrhea | 74 | 11 | 28 | 2 |
| Nausea | 50 | 4 | 28 | <1 |
| Vomiting | 32 | 2 | 14 | <1 |
| Stomatitis | 22 | 2 | 24 | 2 |
| Constipation | 25 | <1 | 19 | <1 |
| Abdominal pain ³ | 23 | 4 | 13 | 2 |
| Dyspepsia | 12 | <1 | 5 | 0 |
| General Disorders and | | | | |
| Administration Site Conditions | | | | |
| Fatigue | 56 | 9 | 47 | 7 |
| Mucosal inflammation | 19 | <1 | 23 | 3 |
| Asthenia | 19 | 4 | 16 | 2 |
| Metabolism and Nutrition | | | | |
| Disorders | | | | |
| Decreased appetite | 46 | 3 | 34 | <1 |
| Skin and Subcutaneous Tissue | | | | |
| Disorders | | | | |
| Palmar-plantar erythrodysesthesia | 42 | 8 | 6 | <1 |
| Rash ⁴ | 23 | <1 | 43 | <1 |
| Dry skin | 11 | 0 | 10 | 0 |
| Vascular Disorders | | | | |
| Hypertension ⁵ | 39 | 16 | 8 | 3 |
| Investigations | | | | |
| Weight decreased | 31 | 2 | 12 | 0 |
| Nervous System Disorders | | | | |
| Dysgeusia | 24 | 0 | 9 | 0 |
| Headache | 11 | <1 | 12 | <1 |
| Dizziness | 11 | 0 | 7 | 0 |
| Endocrine Disorders | | | | |
| Hypothyroidism | 21 | 0 | <1 | <1 |
| Respiratory, Thoracic, and | | | | |
| Mediastinal Disorders | | | | |
| Dysphonia | 20 | <1 | 4 | 0 |
| Dyspnea | 19 | 3 | 29 | 4 |
| Cough | 18 | <1 | 33 | <1 |
| Blood and Lymphatic Disorders | | | | |
| Anemia | 17 | 5 | 38 | 16 |
| Musculoskeletal and Connective | | | | |
| Tissue Disorders | | | | |
| Pain in extremity | 14 | 1 | 8 | <1 |
| Muscle spasms | 13 | 0 | 5 | 0 |
| Arthralgia | 11 | <1 | 14 | 1 |
| Renal and Urinary Disorders | | | | |
| Proteinuria | 12 | 2 | 9 | <1 |

¹ One subject randomized to everolimus received cabozantinib.
²National Cancer Institute Common Terminology Criteria for Adverse Events Version 4.0
³ Includes PT terms abdominal pain, abdominal pain upper, and abdominal pain lower
⁴ Includes PT terms rash, rash erythematous, rash follicular, rash macular, rash papular, rash pustular, rash vesicular, genital rash, intermittent leg rash, rash on scrotum and penis, rash maculo-papular, rash pruritic, contact dermatitis, dermatitis acneiform
⁵ Includes PT terms hypertension, blood pressure increased, hypertensive crisis, blood pressure fluctuation

Other clinically important adverse reactions (all grades) that were reported in <10% of patients treated with CABOMETYX included: wound complications (2%), convulsion (<1%), pancreatitis (<1%), osteonecrosis of the jaw (<1%), and hepatitis cholestatic (<1%).

TABLE 10
Laboratory Abnormalities Occurring in ≥25%
Patients Who Received CABOMETYX in METEOR

| | CABOMETYX | | Everolimus | |
|---|---|---|---|---|
| | (n = 331) | | (n = 322) | |
| | All | Grade | All | Grade |
| | Grades | 3-4 | Grades | 3-4 |
| Test | Percentage (%) of Patients | | | |
| Chemistry | | | | |
| AST increased | 74 | 3 | 40 | <1 |
| ALT increased | 68 | 3 | 32 | <1 |
| Creatinine increased | 58 | <1 | 71 | 0 |
| Triglycerides increased | 53 | 4 | 73 | 13 |
| Hypophosphatemia | 48 | 8 | 36 | 5 |
| Hyperglycemia | 37 | 2 | 59 | 8 |
| Hypoalbuminemia | 36 | 2 | 28 | <1 |
| ALP increased | 35 | 2 | 29 | 1 |
| Hypomagnesemia | 31 | 7 | 4 | <1 |
| Hyponatremia | 30 | 8 | 26 | 6 |
| GGT increased | 27 | 5 | 43 | 9 |
| Hematology | | | | |
| White blood cells decreased | 35 | <1 | 31 | <1 |
| Absolute neutrophil count | 31 | 2 | 17 | <1 |
| decreased | | | | |
| Hemoglobin decreased | 31 | 4 | 71 | 17 |
| Lymphocytes decreased | 25 | 7 | 39 | 12 |
| Platelets decreased | 25 | <1 | 27 | <1 |

ALP, alkaline phosphatase;
ALT, alanine aminotransferase;
AST, aspartate aminotransferase;
GGT, gamma glutamyl transferase.
National Cancer Institute Common Terminology Criteria for Adverse Events, Version 4.0

### CABOSUN

The safety of CABOMETYX was evaluated in CABOSUN, a randomized, open-label trial in patients with advanced renal cell carcinoma, in which 78 patients received 60 mg CABOMETYX daily and 72 patients received 50 mg sunitinib taken once daily (4 weeks on treatment followed by 2 weeks off), until disease progression or unacceptable toxicity [see Clinical Studies (14)]. The median duration of treatment was 6.5 months (range 0.2-28.7) for patients receiving CABOMETYX and 3.1 months (range 0.2-25.5) for patients receiving sunitinib.

Within 30 days of treatment, there were 4 deaths in patients treated with CABOMETYX and 6 deaths in patients treated with sunitinib. Of the 4 patients treated with CABOMETYX, two patients died due to gastrointestinal perforation, one patient had acute renal failure, and one patient died due to clinical deterioration. All Grade 3-4 adverse reactions were collected in the entire safety population. The most frequent Grade 3-4 adverse reactions (≥5%) in patients treated with CABOMETYX were hypertension, diarrhea, hyponatremia, hypophosphatemia, PPE, fatigue, ALT increased, decreased appetite, stomatitis, pain, hypotension, and syncope.

The median average daily dose was 50.3 mg for CABOMETYX and 44.7 mg for sunitinib (excluding scheduled sunitinib non-dosing days). The dose was reduced in 46% of patients receiving CABOMETYX and in 35% of patients receiving sunitinib. The dose was held in 73% of patients receiving CABOMETYX and in 71% of patients receiving sunitinib. Based on patient disposition, 21% of patients receiving CABOMETYX and 22% of patients receiving sunitinib discontinued due to an adverse reaction.

TABLE 11
Grade 3-4 Adverse Reactions Occurring in ≥1%
Patients Who Received CABOMETYX in CABOSUN

| | CABOMETYX | Sunitinib |
|---|---|---|
| | (n = 78) | (n = 72) |
| | Grade 3-4¹ | Grade 3-4¹ |
| | Percentage (%) of Patients | |
| Patients with any Grade 3-4 Adverse | 68 | 65 |
| Reaction | | |
| Gastrointestinal Disorders | | |
| Diarrhea | 10 | 11 |
| Stomatitis | 5 | 6 |
| Nausea | 3 | 4 |
| Vomiting | 1 | 3 |
| Constipation | 1 | 0 |
| General Disorders and Administration Site | | |
| Conditions | | |
| Fatigue | 6 | 17 |
| Pain | 5 | 0 |
| Metabolism and Nutrition Disorders | | |
| Hyponatremia² | 9 | 8 |
| Hypophosphatemia² | 9 | 7 |
| Decreased appetite | 5 | 1 |
| Dehydration | 4 | 1 |
| Hypocalcemia² | 3 | 0 |
| Hypomagnesemia² | 3 | 0 |
| Hyperkalemia² | 1 | 3 |
| Skin and Subcutaneous Skin Disorders | | |
| Palmar-plantar erythrodysesthesia | 8 | 4 |
| Skin ulcer | 3 | 0 |
| Vascular Disorders | | |
| Hypertension³ | 28 | 21 |
| Hypotension | 5 | 1 |
| Angiopathy | 1 | 1 |
| Investigations | | |
| ALT increased² | 5 | 0 |
| Weight decreased | 4 | 0 |
| AST increased² | 3 | 3 |
| Blood creatinine increased² | 3 | 3 |
| Lymphocyte count decreased² | 1 | 6 |
| Platelet count decreased² | 1 | 11 |
| Nervous System Disorders | | |
| Syncope | 5 | 0 |
| Respiratory, Thoracic, and Mediastinal | | |
| Disorders | | |
| Dyspnea | 1 | 6 |
| Dysphonia | 1 | 0 |
| Blood and Lymphatic Disorders | | |
| Anemia | 1 | 3 |
| Psychiatric Disorders | | |
| Depression | 4 | 0 |
| Confusional state | 1 | 1 |
| Infections and Infestations | | |
| Lung infection | 4 | 0 |
| Musculoskeletal and Connective Tissue | | |
| Disorders | | |
| Back pain | 4 | 0 |
| Bone pain | 3 | 1 |
| Pain in extremity | 3 | 0 |
| Arthralgia | 1 | 0 |
| Renal and Urinary Disorders | | |
| Renal failure acute | 4 | 1 |
| Proteinuria | 3 | 1 |

ALT, alanine aminotransferase;
AST, aspartate aminotransferase
¹National Cancer Institute Common Terminology Criteria for Adverse Events Version 4.0
²Laboratory abnormalities are reported as adverse reactions and not based on shifts in laboratory values
³Includes PT term hypertension

### Hepatocellular Carcinoma

### CELESTIAL

The safety of CABOMETYX was evaluated in CELESTIAL, a randomized, double-blinded trial in which 467 patients with advanced hepatocellular carcinoma received 60 mg CABOMETYX and 237 patients received placebo administered daily until disease progression or unacceptable toxicity. Patients on both arms who had disease progression could continue treatment at the discretion of the investigator [see Clinical Studies (14)]. The median duration of treatment was 3.8 months (range 0.1-37.3) for patients receiving CABOMETYX and 2.0 months (range 0.0-27.2) for patients receiving placebo.

Adverse reactions which occurred in ≥25% of CABOMETYX-treated patients included, in order of decreasing frequency: diarrhea, decreased appetite, PPE, fatigue, nausea, hypertension, and vomiting. Grade 3-4 adverse reactions and laboratory abnormalities which occurred in ≥5% of patients were PPE, hypertension, AST increased, fatigue, diarrhea, asthenia, and decreased appetite. Within 30 days of treatment, there were 6 Grade 5 adverse reactions related to CABOMETYX (hepatic failure, hepatorenal syndrome, esophageal fistula, portal vein thrombosis, pulmonary embolism, upper gastrointestinal hemorrhage) and 1 related to placebo (hepatic failure).

The median average daily dose was 35.8 mg for CABOMETYX. The dose was reduced in 62% of patients receiving CABOMETYX and in 13% of patients receiving placebo. Thirty-three percent (33%) of patients received 20 mg CABOMETYX as their lowest dose. The most frequent adverse reactions leading to dose reduction in patients treated with CABOMETYX were: PPE, diarrhea, fatigue, hypertension, and AST increased. Adverse reactions led to study treatment being held in 84% patients receiving CABOMETYX and in 37% patients receiving placebo. Related adverse reactions led to study treatment discontinuation in 16% of patients receiving CABOMETYX and in 3% of patients receiving placebo. The most frequent adverse reactions leading to permanent discontinuation in patients treated with CABOMETYX were PPE (2.4%), fatigue (1.5%), decreased appetite (1.1%), diarrhea (1.1%), and nausea (1.1%).

TABLE 12
Adverse Reactions Occurring at a Higher Incidence in
CABOMETYX-Treated Patients in CELESTIAL (Between-Arm
Difference of ≥5% [All Grades] or ≥2% [Grade 3-4])

| | CABOMETYX | | | | Placebo | | |
|---|---|---|---|---|---|---|---|
| | N = 467 | | | | N = 237 | | |
| | n (%) | | | | n (%) | | |
| | All | | Grade | | All | | Grade |
| | Grades¹ | | 3/4 | | Grades¹ | | 3/4 |
| Preferred Term | Percentage (%) of Patients | | | | | | |
| Gastrointestinal | | | | | | | |
| Disorders | | | | | | | |
| Diarrhea | 251 | (54) | 46 | (9.9) | 44 | (19) | 4 (1-7) |
| Nausea | 147 | (31) | 10 | (2.1) | 42 | (18) | 4 (1-7) |
| Vomiting | 121 | (26) | 2 | (0.4) | 28 | (12) | 6 (2.5) |
| Stomatitis | 63 | (13) | 8 | (1-7) | 5 | (2.1) | 0 |
| Dyspepsia | 47 | (10) | 0 | | 7 | (3.0) | 0 |
| General Disorders and | | | | | | | |
| Administration Site | | | | | | | |
| Conditions | | | | | | | |
| Fatigue | 212 | (45) | 49 | (10) | 70 | (30) | 10 (4.2) |
| Asthenia | 102 | (22) | 32 | (6.9) | 18 | (7.6) | 4 (1-7) |
| Mucosal inflammation | 65 | (14) | 8 | (1-7) | 5 | (2.1) | 1 (0.4) |
| Metabolism and | | | | | | | |
| Nutrition Disorders | | | | | | | |
| Decreased appetite | 225 | (48) | 27 | (5.8) | 43 | (18) | 1 (0.4) |
| Skin and Subcutaneous | | | | | | | |
| Tissue Disorders | | | | | | | |
| Palmar-plantar | 217 | (46) | 79 | (17) | 12 | (5.1) | 0 |
| erythrodysesthesia | | | | | | | |
| Rash | 58 | (12) | 2 | (0.4) | 14 | (5.9) | 1 (0.4) |
| Vascular Disorders | | | | | | | |
| Hypertension | 137 | (29) | 74 | (16) | 14 | (5.9) | 4 (1-7) |
| Investigations | | | | | | | |
| Weight decreased | 81 | (17) | 5 | (1-1) | 14 | (5.9) | 0 |
| Nervous System | | | | | | | |
| Disorders | | | | | | | |
| Dysgeusia | 56 | (12) | 0 | | 5 | (2.1) | 0 |
| Endocrine Disorders | | | | | | | |
| Hypothyroidism | 38 | (8.1) | 2 | (0.4) | 1 | (0-4) | 0 |
| Respiratory, Thoracic, | | | | | | | |
| and Mediastinal | | | | | | | |
| Disorders | | | | | | | |
| Dysphonia | 90 | (19) | 3 | (0.6) | 5 | (2.1) | 0 |
| Dyspnea | 58 | (12) | 15 | (3.2) | 24 | (10) | 1 (0.4) |
| Musculoskeletal and | | | | | | | |
| Connective Tissue | | | | | | | |
| Disorders | | | | | | | |
| Pain in extremity | 44 | (9.4) | 2 | (0.4) | 9 | (3.8) | 2 (0.8) |
| Muscle spasms | 39 | (8.4) | 1 | (0.2) | 4 | (1-7) | 0 |

TABLE 13
Laboratory Abnormalities Occurring at a Higher Incidence
in CABOMETYX-Treated Patients in CELESTIAL (Between-Arm
Difference of ≥5% [All Grades] or ≥2% [Grade 3-4])

| | CABOMETYX | | | | Placebo | | | |
|---|---|---|---|---|---|---|---|---|
| | N = 467 | | | | N = 237 | | | |
| | n (%) | | | | n (%) | | | |
| | All | | Grade | | All | | Grade | |
| | Grades | | 3/4 | | Grades | | 3/4 | |
| Abnormality | Percentage of Patients | | | | | | | |
| Chemistry | | | | | | | | |
| LDH increased | 390 | (84) | 42 | (9.0) | 69 | (29) | 5 | (2.1) |
| ALT increased | 340 | (73) | 58 | (12) | 87 | (37) | 14 | (5.9) |
| AST increased | 343 | (73) | 112 | (24) | 108 | (46) | 45 | (19) |
| Hypoalbu- | 236 | (51) | 6 | (1-3) | 75 | (32) | 3 | (1-3) |
| minemia | | | | | | | | |
| ALP increased | 200 | (43) | 36 | (7.7) | 91 | (38) | 15 | (6.3) |
| Hypophos- | 118 | (25) | 41 | (8.8) | 20 | (8.4) | 9 | (3.8) |
| phatemia | | | | | | | | |
| Hypokalemia | 106 | (23) | 26 | (5.6) | 15 | (6.3) | 3 | (1-3) |
| Hypomagnesemia | 104 | (22) | 15 | (3.2) | 6 | (2.5) | 0 | |
| Amylase | 73 | (16) | 9 | (1-9) | 21 | (8.9) | 5 | (2.1) |
| increased | | | | | | | | |
| Hypocalcemia | 36 | (7.7) | 8 | (1-7) | 0 | | 0 | |
| Hematology | | | | | | | | |
| Platelets | 252 | (54) | 49 | (10) | 37 | (16) | 3 | (1-3) |
| decreased | | | | | | | | |
| White blood cells | 239 | (51) | 23 | (4.9) | 31 | (13) | 2 | (0.8) |
| decreased | | | | | | | | |
| Absolute | 200 | (43) | 32 | (6.9) | 20 | (8.4) | 3 | (1-3) |
| neutrophil count | | | | | | | | |
| decreased | | | | | | | | |

| | | | | | | |
|---|---|---|---|---|---|---|
| Hemoglobin | 36 | (7.7) | 0 | 2 | (0.8) | 0 |
| increased | | | | | | |

ALP, alkaline phosphatase;
ALT, alanine aminotransferase;
AST, aspartate aminotransferase;
LDH, blood lactate dehydrogenase

### 7 Drug Interactions

TABLE 14
Clinically Significant Drug Interactions
Involving Drugs that Affect Cabozantinib
Strong CYP3A4 Inhibitors

| | |
|---|---|
| Clinical | Concomitant use of CABOMETYX with a strong |
| Implications: | CYP3A4 inhibitor increased the exposure of |
| | cabozantinib compared to the use of CABOMETYX |
| | alone [see Clinical Pharmacology (12.3)]. |
| | Increased cabozantinib exposure may increase the risk of |
| | exposure-related toxicity. |
| Prevention or | Reduce the dosage of CABOMETYX if concomitant use |
| Management: | with strong CYP3A4 inhibitors cannot be avoided [see |
| | Dosage and Administration (2.2)]. |

Strong CYP3A4 Inhibitors

| | |
|---|---|
| Examples: | Boceprevir, clarithromycin, conivaptan, grapefruit juice^{a}, |
| | indinavir, itraconazole, ketoconazole, lopinavir/ritonavir, |
| | nefazodone, nelfinavir, posaconazole, ritonavir, |
| | saquinavir, telithromycin, and voriconazole |

Strong CYP3A4 Inducers

| | |
|---|---|
| Clinical | Concomitant use of CABOMETYX with a strong |
| Implications: | CYP3A4 inducer decreased the exposure of |
| | cabozantinib compared to the use of CABOMETYX |
| | alone [see Clinical Pharmacology (12.3)]. |
| | Decreased cabozantinib exposure may lead to reduced |
| | efficacy. |
| Prevention or | Increase the dosage of CABOMETYX if concomitant use |
| Management: | with strong CYP3A4 inducers cannot be avoided [see |
| | Dosage and Administration (2.2)]. |
| Examples: | Rifampin, phenytoin, carbamazepine, phenobarbital, |
| | rifabutin, rifapentine, and St. John's Wort^{b} |

^{a}The effect of grapefruit juice varies widely among brands and is concentration-, dose-, and preparation dependent. Studies have shown that it can be classified as a “strong CYP3A inhibitor” when a certain preparation was used (e.g., high dose, double strength) or as a “moderate CYP3A inhibitor” when another preparation was used (e.g., low dose, single strength).
^{b}The effect of St. John's Wort varies widely and is preparation-dependent

### 8 Use in Specific Populations

### 8.1 Pregnancy

### Risk Summary

Based on findings from animal studies and its mechanism of action, CABOMETYX can cause fetal harm when administered to a pregnant woman [see Clinical Pharmacology (12.1)]. There are no available data in pregnant women to inform the drug-associated risk. In animal developmental and reproductive toxicology studies administration of cabozantinib to pregnant rats and rabbits during organogenesis resulted in embryofetal lethality and structural anomalies at exposures that were below those occurring clinically at the recommended dose [see Nonclinical Toxicology (13.1)]. Advise pregnant women or women of childbearing potential of the potential hazard to a fetus.

The estimated background risk of major birth defects and miscarriage for the indicated population is unknown. In the U.S. general population, the estimated background risk of major birth defects and miscarriage in clinically recognized pregnancies is 2-4% and 15-20%, respectively.

### Data

### Animal Data

In an embryo-fetal development study in pregnant rats, daily oral administration of cabozantinib throughout organogenesis caused increased embryo-fetal lethality compared to controls at a dose of 0.03 mg/kg (approximately 0.12-fold of human AUC at the recommended dose). Findings included delayed ossification and skeletal variations at a dose of 0.01 mg/kg/day (approximately 0.04-fold of human AUC at the recommended dose).

In pregnant rabbits, daily oral administration of cabozantinib throughout organogenesis resulted in findings of visceral malformations and variations including reduced spleen size and missing lung lobe at 3 mg/kg (approximately 1.1-fold of the human AUC at the recommended dose).

In a pre- and postnatal study in rats, cabozantinib was administered orally from gestation day 10 through postnatal day 20. Cabozantinib did not produce adverse maternal toxicity or affect pregnancy, parturition or lactation of female rats, and did not affect the survival, growth or postnatal development of the offspring at doses up to 0.3 mg/kg/day (0.05-fold of the maximum recommended clinical dose).

### 8.2 Lactation

### Risk Summary

There is no information regarding the presence of cabozantinib or its metabolites in human milk, or their effects on the breastfed infant, or milk production. Because of the potential for serious adverse reactions in a breastfed infant from CABOMETYX, advise a lactating woman not to breastfeed during treatment with CABOMETYX and for 4 months after the final dose.

### 8.3 Females and Males of Reproductive Potential

### Contraception

### Females

CABOMETYX can cause fetal harm when administered to a pregnant woman [see Use in Specific Populations (8.1)]. Advise females of reproductive potential to use effective contraception during treatment with CABOMETYX and for 4 months after the final dose.

### Infertility

### Females and Males

Based on findings in animals, CABOMETYX may impair fertility in females and males of reproductive potential [see Nonclinical Toxicology (13.1)].

### 8.4 Pediatric Use

The safety and effectiveness of CABOMETYX in pediatric patients have not been established.

### Juvenile Animal Data

Juvenile rats were administered cabozantinib daily at doses of 1 or 2 mg/kg/day from Postnatal Day 12 (comparable to less than 2 years in humans) through Postnatal Day 35 or 70. Mortalities occurred at doses equal and greater than 1 mg/kg/day (approximately 0.16 times the clinical dose of 60 mg/day based on body surface area). Hypoactivity was observed at both doses tested on Postnatal Day 22. Targets were generally similar to those seen in adult animals, occurred at both doses, and included the kidney (nephropathy, glomerulonephritis), reproductive organs, gastrointestinal tract (cystic dilatation and hyperplasia in Brunner's gland and inflammation of duodenum; and epithelial hyperplasia of colon and cecum), bone marrow (hypocellularity and lymphoid depletion), and liver. Tooth abnormalities and whitening as well as effects on bones including reduced bone mineral content and density, physeal hypertrophy, and decreased cortical bone also occurred at all dose levels. Recovery was not assessed at the 2 mg/kg dose level (approximately 0.32 times the clinical dose of 60 mg based on body surface area) due to high levels of mortality. At the low dose level, effects on bone parameters were partially resolved but effects on the kidney and epididymis/testis persisted after treatment ceased.

### 8.5 Geriatric Use

In RCC studies, 41% of patients treated with CABOMETYX were age 65 years and older, and 8% of patients were 75 years and older. In the HCC study, 49% of patients were treated with CABOMETYX were age 65 years and older, and 15% of patients were 75 years and older.

In RCC studies, Grade 3-4 adverse reactions occurred in 73% of patients age 65 years and older, and in 76% of patients 75 years and older. In the HCC study, Grade 3-4 adverse reactions occurred in patients age 65 years and older, and in of patients 75 years and older. No overall differences in safety or efficacy were observed between older and younger patients.

### 8.6 Hepatic Impairment

No dose adjustment is recommended in patients with mild (total bilirubin≤ULN and AST>ULN, or total bilirubin>1.0 to ≤1.5×ULN) or moderate (total bilirubin>1.5 to ≤3×ULN and any AST value) hepatic impairment. CABOMETYX is not recommended for use in patients with severe hepatic impairment (total bilirubin>3×ULN and any AST value), since it has not been studied in this population [see Clinical Pharmacology (12.3)]

### 8.7 Renal Impairment

Dosage adjustment is not required in patients with mild or moderate renal impairment. There is no experience with CABOMETYX in patients with severe renal impairment [see Clinical Pharmacology (12.3).]

### 10 Overdosage

One case of overdosage was reported in the cabozantinib clinical program; a patient inadvertently took twice the intended dose (200 mg daily) of another formulation of cabozantinib product for nine days. The patient suffered Grade 3 memory impairment, Grade 3 mental status changes, Grade 3 cognitive disturbance, Grade 2 weight loss, and Grade 1 increase in BUN. The extent of recovery was not documented.

### 11 Description

CABOMETYX is the (S)-malate salt of cabozantinib, a kinase inhibitor. Cabozantinib (S)-malate is described chemically as N-(4-(6,7-dimethoxyquinolin-4-yloxy)phenyl)-N′-4-fluorophenyl)cyclopropane-1,1-dicarboxamide, (2S)-hydroxybutanedioate. The molecular formula is C₂₈H₂₄FN₃O₅.C₄H₆O₅ and the molecular weight is 635.6 Daltons as malate salt. The chemical structure of cabozantinib (S)-malate salt is:

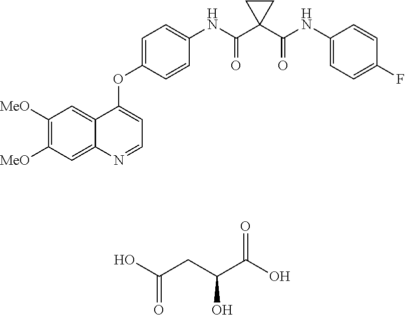 [SMILES: COc1cc2nccc(Oc3ccc(NC(=O)C4(C(=O)Nc5ccc(F)cc5)CC4)cc3)c2cc1OC ; O=C(O)C[C@H](O)C(=O)O]

Cabozantinib (S)-malate salt is a white to off-white solid that is practically insoluble in aqueous media.

CABOMETYX (cabozantinib) tablets for oral use are supplied as film-coated tablets containing 20 mg, 40 mg, or 60 mg of cabozantinib, which is equivalent to 25 mg, 51 mg, or 76 mg of cabozantinib (S)-malate, respectively. CABOMETYX also contains the following inactive ingredients: microcrystalline cellulose, lactose anhydrous, hydroxypropyl cellulose, croscarmellose sodium, colloidal silicon dioxide, and magnesium stearate.

The film coating contains hypromellose, titanium dioxide, triacetin, and iron oxide yellow.

### 12 Clinical Pharmacology

### 12.1 Mechanism of Action

In vitro biochemical and/or cellular assays have shown that cabozantinib inhibits the tyrosine kinase activity of MET, VEGFR-1, -2 and -3, AXL, RET, ROS1, TYRO3, MER, KIT, TRKB, FLT-3, and TIE-2. These receptor tyrosine kinases are involved in both normal cellular function and pathologic processes such as oncogenesis, metastasis, tumor angiogenesis, drug resistance, and maintenance of the tumor microenvironment.

### 12.2 Pharmacodynamics

The exposure-response or -safety relationship for cabozantinib is unknown.

### Cardiac Electrophysiology

The effect of orally administered cabozantinib on QTc interval was evaluated in a randomized, double-blinded, placebo-controlled study in patients with medullary thyroid cancer administered a dose of 140 mg. A mean increase in QTcF of 10-15 ms was observed at 4 weeks after initiating cabozantinib. A concentration-QTc relationship could not be definitively established. Changes in cardiac wave form morphology or new rhythms were not observed. No cabozantinib-treated patients in this study had a confirmed QTcF>500 ms nor did any cabozantinib-treated patients in the RCC or HCC studies (at a dose of 60 mg).

### 12.3 Pharmacokinetics

Repeat daily dosing of cabozantinib at 140 mg for 19 days resulted in 4- to 5-fold mean cabozantinib accumulation (based on AUC) compared to a single dose administration; steady state was achieved by Day 15.

### Absorption

Following oral administration of cabozantinib, median time to peak cabozantinib plasma concentrations (Tₘₐₓ) ranged from 3 to 4 hours post-dose.

A 19% increase in the Cₘₐₓ of the tablet formulation (CABOMETYX) compared to the capsule formulation (COMETRIQ®) was observed following a single 140 mg dose. A less than 10% difference in the AUC was observed between cabozantinib tablet (CABOMETYX) and capsule (COMETRIQ) formulations [see Dosage and Administration (2.1)].

Cabozantinib Cₘₐₓ and AUC values increased by 41% and 57%, respectively, following a high-fat meal relative to fasted conditions in healthy subjects administered a single 140 mg oral dose of an investigational cabozantinib capsule formulation.

### Distribution

The oral volume of distribution (V_{Z}/F) of cabozantinib is approximately 319 L. Cabozantinib is highly protein bound in human plasma (≥99.7%).

### Elimination

The predicted terminal half-life is approximately 99 hours and the clearance (CL/F) at steady-state is estimated to be 2.2 L/hr.

### Metabolism

Cabozantinib is a substrate of CYP3A4 in vitro.

### Excretion

Approximately 81% of the total administered radioactivity was recovered within a 48-day collection period following a single 140 mg dose of an investigational ¹⁴C-cabozantinib formulation in healthy subjects. Approximately 54% was recovered in feces and 27% in urine. Unchanged cabozantinib accounted for 43% of the total radioactivity in feces and was not detectable in urine following a 72 hour collection.

### Specific Populations

The following patient characteristics did not result in a clinically relevant difference in the pharmacokinetics of cabozantinib: age (32-86 years), sex, race (Whites and non-Whites), or mild to moderate renal impairment (eGFR greater than or equal to 30 mL/min/1.73 m² as estimated by MDRD (modification of diet in renal disease equation)). The pharmacokinetics of cabozantinib is unknown in patients with worse than moderate renal impairment (eGFR less than 29 mL/min/1.73 m²) as estimated by MDRD equation or renal impairment requiring dialysis.

### Hepatic Impairment

Based on an integrated population pharmacokinetic analysis of cabozantinib in healthy subjects and cancer patients (including HCC), no clinically significant differences in the mean cabozantinib plasma exposure were observed amongst subjects with normal liver function (total bilirubin and AST≤ULN, n=1425), mild hepatic impairment (total bilirubin≤ULN and AST>ULN or total bilirubin>1.0 to 1.5×ULN and any AST value, n=558), and moderate hepatic impairment (total bilirubin>1.5 to ≤3×ULN and any AST value, n=15). The pooled analysis included 391 patients with HCC of whom 128, 308, and 11 were categorized as having normal liver function, mild, and moderate hepatic impairment, respectively. The pharmacokinetics of cabozantinib were not evaluated in patients with severe hepatic impairment (total bilirubin>3×ULN and any AST value) [see Use in Specific Populations (8.6)].

### Pediatric Population

The pharmacokinetics of cabozantinib has not been established in the pediatric population [see Use in Specific Populations (8.4)].

### Drug Interactions

### CYP3A4 Inhibition on Cabozantinib

Administration of a strong CYP3A4 inhibitor, ketoconazole (400 mg daily for 27 days) to healthy subjects increased single-dose plasma cabozantinib exposure (AUC_{0-inf}) by 38%.

### CYP3A4 Induction on Cabozantinib

Administration of a strong CYP3A4 inducer, rifampin (600 mg daily for 31 days) to healthy subjects decreased single-dose plasma cabozantinib exposure (AUC_{0-inf}) by 77%.

### Cabozantinib on CYP2C8 Substrates

No clinically-significant effect on single-dose rosiglitazone (a CYP2C8 substrate) plasma exposure (Cₘₐₓ and AUC) was observed when co-administered with cabozantinib at steady-state plasma concentrations (≥100 mg/day daily for a minimum of 21 days) in patients with solid tumors.

### Gastric pH Modifying Agents on Cabozantinib

No clinically-significant effect on plasma cabozantinib exposure (AUC) was observed following co-administration of the proton pump inhibitor (PPI) esomeprazole (40 mg daily for 6 days) with a single dose of 100 mg cabozantinib to healthy volunteers.

### In Vitro Studies

### Metabolic Pathways

Inhibition of CYP3A4 reduced the formation of the oxidative metabolite by >80%. Inhibition of CYP2C9 had a minimal effect on cabozantinib metabolite formation (i.e., a <20% reduction). Inhibition of CYP1A2, CYP2A6, CYP2B6, CYP2C8, CYP2C19, CYP2D6 and CYP2E1 had no effect on cabozantinib metabolite formation.

Although cabozantinib is an inhibitor of CYP2C8 in vitro, a clinical study of this potential interaction concluded that concurrent use did not result in a clinically relevant effect on CYP2C8 substrate exposure. Given this finding, other less sensitive substrates of pathways affected by cabozantinib in vitro (i.e., CYP2C9, CYP2C19, and CYP3A4) were not evaluated in a clinical study because, although a clinically relevant exposure effect cannot be ruled out, it is unlikely. Cabozantinib does not inhibit CYP1A2 and CYP2D6 isozymes in vitro.

Cabozantinib is an inducer of CYP1A1 mRNA; however, the clinical relevance of this finding is unknown. Cabozantinib does not induce CYP1A2, CYP2B6, CYP2C8, CYP2C9, CYP2C19 or CYP3A4.

### Drug Transporter Systems

Cabozantinib is an inhibitor, but not a substrate, of P-gp transport activities and has the potential to increase plasma concentrations of co-administered substrates of P-gp. The clinical relevance of this finding is unknown.

Cabozantinib is a substrate of MRP2 in vitro and MRP2 inhibitors have the potential to increase plasma concentrations of cabozantinib. The clinical relevance of this finding is unknown.

### 13 Nonclinical Toxicology

### 13.1 Carcinogenesis, Mutagenesis, Impairment of Fertility

The carcinogenic potential of cabozantinib has been evaluated in two species: rasH2 transgenic mice and Sprague-Dawley rats. In the 2-year rat carcinogenicity study, once daily oral administration of cabozantinib resulted in a statistically significant increase in the incidence of malignant/complex malignant pheochromocytoma in combination with benign pheochromocytoma or in benign pheochromocytoma alone in male rats at a dose of 1 mg/kg (approximately 5 times the human exposure by AUC at the recommended 60 mg dose).

Cabozantinib was not carcinogenic in a 26-week carcinogenicity study in rasH2 transgenic mice at a slightly higher exposure than the intended human therapeutic exposure.

Cabozantinib was not mutagenic in vitro in the bacterial reverse mutation (Ames) assay and was not clastogenic in both the in vitro cytogenetic assay using human lymphocytes or in the in vivo mouse micronucleus assay.

Based on nonclinical findings, male and female fertility may be impaired by treatment with CABOMETYX. In a fertility study in which cabozantinib was administered to male and female rats at doses of 1, 2.5, and 5 mg/kg/day, male fertility was significantly compromised at doses equal to or greater than 2.5 mg/kg/day (approximately 13-fold of human AUC at the recommended dose), with a decrease in sperm counts and reproductive organ weights. In females, fertility was significantly reduced at doses equal to or greater than 1 mg/kg/day (5-fold of human AUC at the recommended dose) with a significant decrease in the number of live embryos and a significant increase in pre- and post-implantation losses.

Observations of effects on reproductive tract tissues in general toxicology studies were supportive of effects noted in the dedicated fertility study and included hypospermia and absence of corpora lutea in male and female dogs in a 6-month repeat dose study at plasma exposures (AUC) approximately 0.5-fold (males) and ≤0.1-fold (females) of those expected in humans at the recommended dose. In addition, female rats administered 5 mg/kg/day for 14 days (approximately 9-fold of human AUC at the recommended dose) exhibited ovarian necrosis.

### 14 Clinical Studies

### 14.1 Renal Cell Carcinoma

### METEOR

METEOR (NCT01865747) was a randomized (1:1), open-label, multicenter study of CABOMETYX versus everolimus conducted in patients with advanced RCC who had received at least 1 prior anti-angiogenic therapy. Patients had to have a Karnofsky Performance Score (KPS)≥70%. Patients were stratified by the number of prior VEGFR tyrosine kinase inhibitors (TKIs) and Memorial Sloan Kettering Cancer Center (MSKCC) Risk Group.

Patients (N=658) were randomized to receive CABOMETYX (N=330) administered orally at 60 mg daily or everolimus (N=328) administered orally at 10 mg daily. The majority of the patients were male (75%), with a median age of 62 years. Sixty-nine percent (69%) received only one prior anti-angiogenic therapy. Patient distribution by MSKCC risk groups was 46% favorable (0 risk factors), 42% intermediate (1 risk factor), and 13% poor (2 or 3 risk factors). Fifty-four percent (54%) of patients had 3 or more organs with metastatic disease, including lung (63%), lymph nodes (62%), liver (29%), and bone (22%).

The main efficacy outcome measure was progression-free survival (PFS) assessed by a blinded independent radiology review committee among the first 375 subjects randomized. Other efficacy endpoints were objective response rate (ORR) and overall survival (OS) in the Intent-to-Treat (ITT) population. Tumor assessments were conducted every 8 weeks for the first 12 months, then every 12 weeks thereafter. Patients received treatment until disease progression or experiencing unacceptable toxicity. Patients on both arms who had disease progression could continue treatment at the discretion of the investigator.

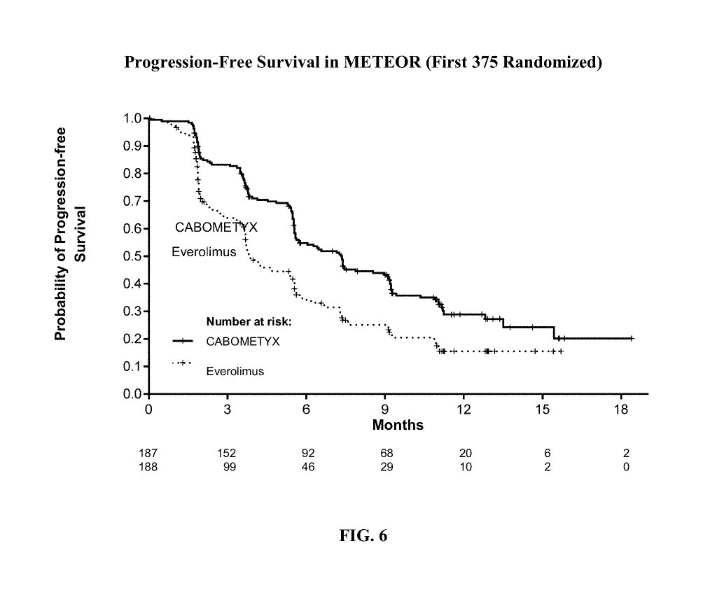

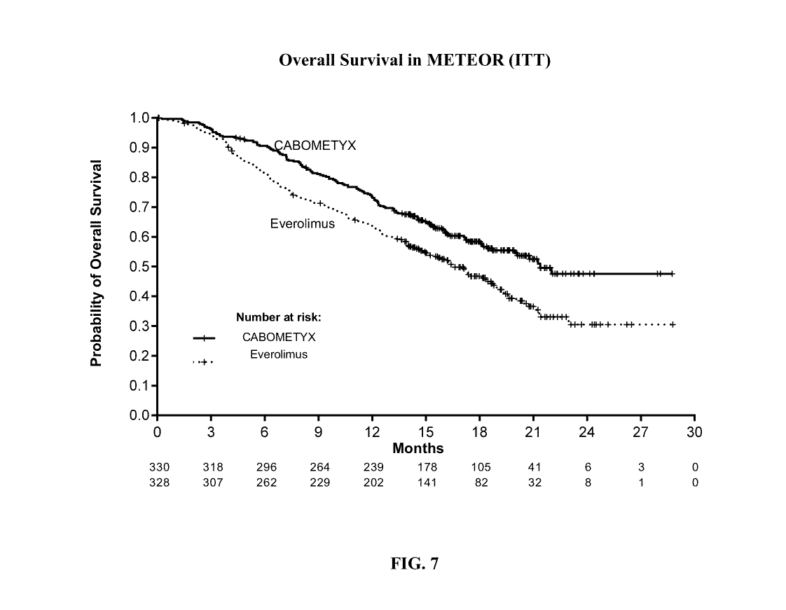

Statistically significant improvements in PFS, OS, and ORR were demonstrated for CABOMETYX compared to everolimus (FIGS. 6 and 7 and Tables 15 and 16).

TABLE 15
Progression-Free Survival in METEOR (First 375 Randomized)

| | CABOMETYX | Everolimus |
|---|---|---|
| Endpoint | N = 187 | N = 188 |
| Median PFS (95% CI), months | 7.4 (5.6, 9.1) | 3.8 (3.7, 5.4) |

| | |
|---|---|
| HR (95% CI), p-value¹ | 0.58 (0.45, 0.74), p < 0.0001 |

¹stratified log-rank test with prior VEGFR-targeting TKI therapy (1 vs 2 or more) and MSKCC prognostic criteria for previously treated patients with RCC (0 vs 1 vs 2 or 3) as stratification factors (per IVRS data)

TABLE 16
Overall Survival and Objective Response Rate in METEOR (ITT)

| | CABOMETYX | | Everolimus | |
|---|---|---|---|---|
| Endpoint | N = 330 | | N = 328 | |
| Median OS (95% CI), months | 21.4 | (18.7, NE) | 16.5 | (14.7, 18.8) |
| HR (95% CI), p-value¹ | 0.66 (0.53, 0.83), p = 0.0003 | | | |
| Confirmed ORR (partial | 17% | (13%, 22%) | 3% | (2%, 6%) |
| responses only) (95% CI) | | | | |

| | |
|---|---|
| p-value² | p < 0.0001 |

¹stratified log-rank test with prior VEGFR-targeting TKI therapy (1 vs 2 or more) and MSKCC prognostic criteria for previously treated patients with RCC (0 vs 1 vs 2 or 3) as stratification factors (per IVRS data)
²chi-squared test

### CABOSUN

CABOSUN (NCT01835158) was a randomized (1:1), open-label, multicenter study of CABOMETYX versus sunitinib conducted in patients with advanced RCC who had not received prior therapy. Patients were randomized to receive CABOMETYX (N=79) 60 mg orally daily or sunitinib (N=78) 50 mg orally daily (4 weeks on treatment followed by 2 weeks off) until disease progression or unacceptable toxicity. All patients were required to have intermediate or poor risk disease as defined by the International Metastatic RCC Database Consortium (IMDC) risk group categories. Patients were stratified by IMDC risk group and presence of bone metastases (yes/no).

The majority of patients were male (78%), with a median age of 63 years. Patient distribution by IMDC risk groups was 81% intermediate (1-2 risk factors) and 19% poor (≥3 risk factors). Thirty-six percent (36%) patients had bone metastases. Forty-six percent (46%) of patients were ECOG 0, 41% ECOG 1, and 13% ECOG 2.

The major efficacy outcome measure was progression-free survival (PFS) by a retrospective blinded independent radiology review committee (BIRC).

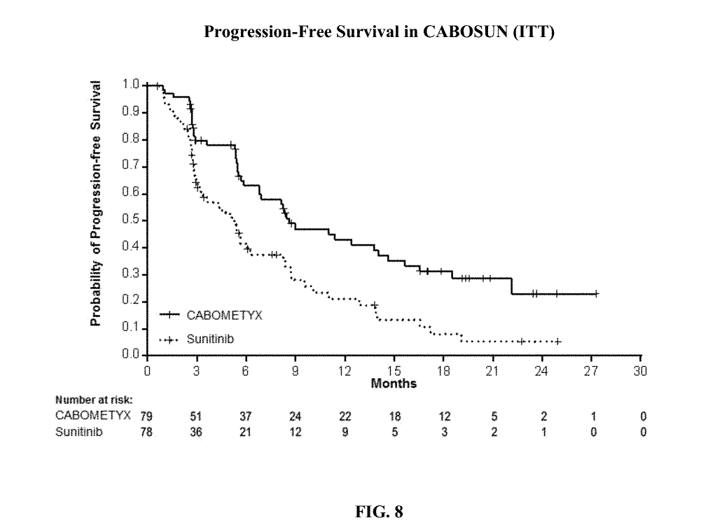

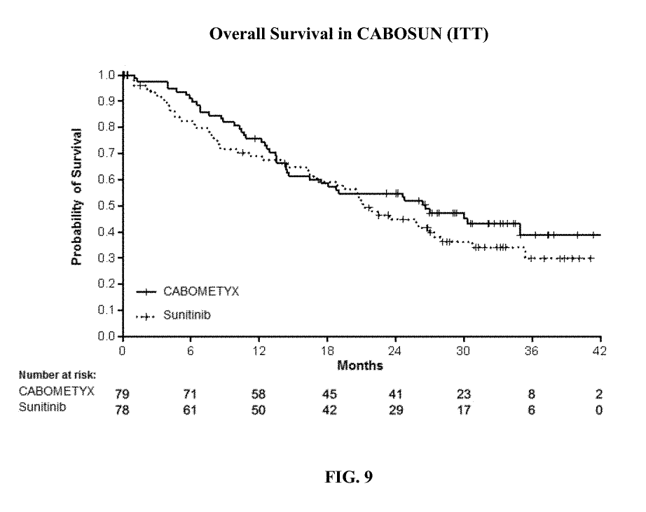

A statistically significant improvement in PFS, as assessed by a blinded independent radiology review committee, was demonstrated for CABOMETYX compared to sunitinib (FIG. 8, Table 17). The OS results are presented in FIG. 9 and Table 17; ORR results are presented in Table 17.

TABLE 17
Progression-free Survival, Overall Survival
and Objective Response Rate in CABOSUN (ITT)

| | CABOMETYX | | Sunitinib | |
|---|---|---|---|---|
| Endpoint | N = 79 | | N = 78 | |
| Progression-Free Survival¹ | | | | |
| Events, n(%) | 43 | (54) | 49 | (63) |
| Median PFS (95% CI), months¹ | 8.6 | (6.8, 14.0) | 5.3 | (3.0, 8.2) |
| Hazard Ratio² (95% CI), p-value³ | 0.48 (0.31, 0.74), p = 0.0008 | | | |
| Overall Survival | | | | |
| Events, n(%) | 43 | (54) | 47 | (60) |
| Hazard Ratio^{2,4} (95% CI) | 0.80 (0.53, 1.21) | | | |
| Confirmed ORR, partial | 20% | (12.0, 30.8) | 9% | (3.7, 17.6) |
| responses only (95% CI)^{1,4} | | | | |

¹as assessed by a retrospective blinded independent radiology review committee (BIRC)
²estimated from stratified Cox proportional hazards model with stratification factors IMDC risk group and presence of bone metastases and treatment as covariate
³two-sided stratified log-rank test with stratification factors IMDC risk group and presence of bone metastases
⁴no multiplicity adjustments were made for overall survival or ORR

### 14.2 Hepatocellular Carcinoma

The safety and efficacy of CABOMETYX were evaluated in a randomized, double-blinded, placebo-controlled Phase 3 study. Patients (N=707) with advanced HCC who had previously received sorafenib and were Child Pugh A were randomized (2:1) to receive CABOMETYX (N=470) or placebo (N=237). Patients could have received one other prior systemic therapy. The primary efficacy endpoint was overall survival (OS). Secondary efficacy endpoints were progression-free survival (PFS) and objective response rate (ORR), as assessed by investigator using RECIST 1.1. Tumor assessments were conducted every 8 weeks.

The baseline demographic and disease characteristics were similar between the CABOMETYX and placebo arms. The majority of the patients were male (82%), with a median age of 64 years. Fifty-three percent (53%) had ECOG performance status 0; 47% were ECOG 1. The etiology for HCC was 38% hepatitis B virus (HBV), 21% hepatitis C virus (HCV), and 40% other (neither HBV nor HCV). Macroscopic vascular invasion or extra-hepatic tumor spread was present in 78% of patients, and 41% had alfa-fetoprotein (AFP) levels≥400 μg/L. Seventy-two percent (72%) received one prior systemic therapy regimen; 27% received two.

A statistically significant improvement in OS, PFS, and ORR was demonstrated for CABOMETYX compared to placebo (Table 18, FIG. 3A-B).

TABLE 18
Overall Survival, Progression-free Survival, and
Objective Response Rate in CELESTIAL (ITT)

| | CABOMETYX | | Placebo | |
|---|---|---|---|---|
| Endpoint | N = 470 | | N = 237 | |
| Overall Survival | | | | |
| Events, n(%) | 317 | (67) | 167 | (70) |
| Median OS (95% CI), months | 10.2 | (9.1, 12.0) | 8.0 | (6.8, 9.4) |
| Hazard Ratio (95% CI)^{1,2} | 0.76 (0.63, 0.92) | | | |
| p-value¹ | p = 0.0049 | | | |
| Progression-Free Survival³ | | | | |
| Events, n(%) | 349 | (74) | 205 | (86) |
| Median PFS (95% CI), months | 5.2 | (4.0, 5.5) | 1.9 | (1.9, 1.9) |
| Hazard Ratio (95% CI)¹ | 0.44 (0.36, 0.52) | | | |
| p-value¹ | p < 0.0001 | | | |
| Confirmed ORR, partial responses | 4% | (2.3, 6.0) | 0.4% | (0.0, 2.3) |
| only (95% CI)³ | | | | |
| p-value^{1, 4} | p = 0.0086 | | | |
| Stable Disease as Best Response, | 282 | (60) | 78 | (33) |
| n(%) | | | | |
| Progressive Disease as Best | 98 | (21) | 131 | (55) |
| Response, n(%) | | | | |

¹stratified log-rank test with etiology of disease (HBV [with or without HCV], HCV [without HBV], or Other), geographic region (Asia, Other Regions), and presence of extrahepatic spread of disease and/or macrovascular invasion (Yes, No) as stratification factors (per IVRS data)
²estimated using the Cox proportional-hazard model
³as assessed by investigator per RECIST 1.1
⁴stratified Cochran-Mantel-Haenszel (CMH) test

### 16 How Supplied/Storage and Handling

CABOMETYX tablets are supplied as follows:

60 mg tablets are yellow film-coated, oval shaped with no score, debossed with “XL” on one side and “60” on the other side of the tablet; available in bottles of 30 tablets: NDC 42388-023-26

40 mg tablets are yellow film-coated, triangle shaped with no score, debossed with “XL” on one side and “40” on the other side of the tablet; available in bottles of 30 tablets: NDC 42388-025-26

20 mg tablets are yellow film-coated, round shaped with no score, debossed with “XL” on one side and “20” on the other side of the tablet; available in bottles of 30 tablets: NDC 42388-024-26

Store CABOMETYX at 20° C. to 25° C. (68° F. to 77° F.); excursions are permitted from 15° C. to 30° C. (59° F. to 86° F.) [see USP Controlled Room Temperature].

### 17 Patient Counseling Information

Advise the patient to read the FDA-approved patient labeling (Patient Information). Inform patients of the following:
Hemorrhage: Instruct patients to contact their healthcare provider to seek immediate medical attention for signs or symptoms of unusual severe bleeding or hemorrhage [see Warnings and Precautions (5.12)].
Gastrointestinal disorders: Advise patients that gastrointestinal disorders such as diarrhea, nausea, vomiting, and constipation may develop during CABOMETYX treatment and to seek immediate medical attention if they experience persistent or severe abdominal pain because cases of gastrointestinal perforation and fistula have been reported in patients taking CABOMETYX [see Warnings and Precautions (5.2)].
Thrombotic Events: Venous and arterial thrombotic events have been reported.

Advise patients to report signs or symptoms of an arterial thrombosis. Venous thromboembolic events including pulmonary embolus have been reported. Advise patients to contact their health care provider if new onset of dyspnea, chest pain, or localized limb edema occurs [see Warnings and Precautions (5.3)].
Hypertension: Inform patients of the signs and symptoms of hypertension. Advise patients to undergo routine blood pressure monitoring and to contact their health care provider if blood pressure is elevated or if they experience signs or symptoms of hypertension [see Warnings and Precautions 0.4)].
Diarrhea: Advise patients to notify their healthcare provider at the first signs of poorly formed or loose stool or an increased frequency of bowel movements [see Warnings and Precautions (5.5)].
Palmar-plantar erythrodysesthesia: Advise patients to contact their healthcare provider for progressive or intolerable rash [see Warnings and Precautions (5.6)].
Wound healing: Patients should be advised to contact their healthcare provider before any planned surgeries, including dental surgery [see Dosage and Administration (2.2)].
Drug interactions: Advise patients to inform their healthcare provider of all prescription or nonprescription medication or herbal products that they are taking.
Embryo-fetal toxicity: Advise females of reproductive potential of the potential risk to a fetus. Advise females to contact their healthcare provider if they become pregnant, or if pregnancy is suspected, during treatment with CABOMETYX [see Warnings and Precautions (5.8), Use in Specific Populations (8.1)].
Females of reproductive potential: Advise patients of reproductive potential to use effective contraception during treatment with CABOMETYX and for at least four months after the final dose of CABOMETYX [Use in Specific Populations (8.3)].
Lactation: Advise women not to breastfeed during treatment with CABOMETYX and for 4 months following the last dose [Use in Specific Populations (8.2)].
Important Administration Information
   Instruct patients not to eat for at least 2 hours before and at least 1 hour after taking CABOMETYX. Instruct patients to not crush CABOMETYX tablets and to take CABOMETYX tablets with a full glass (at least 8 ounces) of water.
   Advise patients not to consume grapefruits or grapefruit juice while taking CABOMETYX.
   [see Dosage and Administration (2.1)]

### Other Embodiments

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A method of treating hepatocellular carcinoma, comprising administering to a patient in need of such treatment, 20 to 60 mg of cabozantinib or a pharmaceutically acceptable salt thereof once daily.

2. A method of treating hepatocellular carcinoma, comprising administering to a patient in need of such treatment, 20, 40, or 60 mg of cabozantinib or a pharmaceutically acceptable salt thereof once daily.

3. The method of claim 2, wherein the patient received prior therapy, wherein the prior therapy is selected from the group consisting of sorafenib, regorafenib, lenvatinib, tivantinib, ramucirumab, Anti-PD-1/PD-L1 therapy, and doxorubicin.

4. The method of claim 3, wherein the patient received prior sorafenib therapy.

5. The method of claim 4, wherein the cabozantinib is administered as cabozantinib (S)-malate.

6. A method of treating hepatocellular carcinoma, comprising administering to a patient in need of such treatment, 20, 40, or 60 mg of cabozantinib or a pharmaceutically acceptable salt thereof once daily, wherein the treatment results in, as compared to placebo, a median overall survival (OS) of greater than nine months, and one or both of a median progression-free survival (PFS) of greater than 4 months, and a median overall response rate (ORR) of greater than 2 percent.

7. The method of claim 6, wherein the patient received prior therapy, wherein the prior therapy is selected from the group consisting of sorafenib, regorafenib, lenvatinib, tivantinib, ramucirumab, Anti-PD-1/PD-L1 therapy, and doxorubicin.

8. The method of claim 7, wherein the patient received prior sorafenib therapy.

9. The method of claim 8, wherein the cabozantinib is administered as cabozantinib (S)-malate.

10. A method of treating hepatocellular carcinoma in a patient who received prior sorafenib therapy, comprising administering to a patient in need of such treatment, 20, 40, or 60 mg of cabozantinib or a pharmaceutically acceptable salt thereof once daily, wherein the treatment results in, as compared to placebo, a median overall survival (OS) of greater than nine months, and one or both of a median progression-free survival (PFS) of greater than 4 months, and a median overall response rate (ORR) of greater than 2 months.

11. The method of claim 10, wherein the cabozantinib is administered as cabozantinib (S)-malate.

12. A method of treating hepatocellular carcinoma and/or inhibiting the proliferation or migration of hepatocellular carcinoma cells, comprising contacting the hepatocellular carcinoma and/or the hepatocellular carcinoma cells with a medicament comprising cabozantinib or a pharmaceutically acceptable salt thereof.

13. The method of claim 12, wherein the hepatocellular carcinoma or hepatocellular carcinoma cells have been previously treated with a therapy selected from the group consisting of sorafenib, regorafenib, lenvatinib, tivantinib, ramucirumab, Anti-PD-1/PD-L1 therapy, and doxorubicin.

14. The method of claim 13, wherein the hepatocellular carcinoma or hepatocellular carcinoma cells have been previously treated with sorafenib.

15. The method of claim 14, wherein the cabozantinib is administered as cabozantinib (S)-malate.

16. The method according to claim 15, wherein said cabozantinib treatment results in a clinically relevant improvement in one or more markers of disease status and progression, one or more selected from the group consisting of: (i) overall survival; (ii) progression-free survival; (iii) overall response rate; and (iv) reduction in metastatic disease.

17. The method according to claim 16 wherein the patient has an overall survival superior or equal to 10 months.

18. The method according to claim 16 wherein the patient has an overall survival superior or equal to 11 months.

19. The method according to claim 16 wherein the patient has a progression free survival superior or equal to 5 months.

20. The method according to claim 16 wherein the patient has a progression free survival superior or equal to 5.5 months.

21. The method according to claim 16 wherein the patient has an overall response rate superior or equal to four percent.

22. The method according to claim 16 wherein the patient has an overall response rate superior or equal to six percent.

23. The method according to claim 16 wherein the patient has an overall survival (OS) of superior or equal to ten months, a progression-free survival (PFS) superior or equal to 5 month, and an overall response rate (ORR) superior or equal to 4 percent.
